(19) United States
(12) Patent Application Publication
(10) Pub. No.: US 2011/0046018 A1
(43) Pub. Date: Feb. 24, 2011

(54) SERS-BASED, SINGLE STEP, REAL-TIME DETECTION OF PROTEIN KINASE AND/OR PHOSPHATASE ACTIVITY

(76) Inventors: Fanqing Frank Chen, Moraga, CA (US); Gang L. Liu, Berkeley, CA (US); Jonathan A. Ellman, Oakland, CA (US)

Correspondence Address: Weaver Austin Villeneuve & Sampson LLP, P.O. BOX 70250, OAKLAND, CA, 94612-0250, US

(21) Appl. No.: 12/746,158

(22) PCT Filed: Dec. 23, 2008
(86) PCT No.: PCT/US08/88195

§ 371 Date: Sep. 20, 2010

Related U.S. Application Data
(60) Provisional application No. 61/018,286, filed on Dec. 31, 2007.
(60) Provisional application No. 61/022,115, filed on Jan. 18, 2008.

(51) Int. Cl. C40B 50/14; C12M 1/34; C12Q 1/48; C12Q 1/42; B44C 1/22

(52) U.S. Cl. 506/30; 435/288.7; 435/15; 435/21; 216/24

(57) ABSTRACT

This invention provides novel compositions and methods for the detection, and/or quantification, of the presence and/or activity of one or more kinases and/or phosphatases. In certain embodiments this invention a device for the detection of kinase and/or phosphatase activity where the device comprises a Raman active surface comprising features that enhance Raman scattering having attached thereto a plurality of kinase and/or phosphatase substrate molecules.

### CROSS-REFERENCE TO RELATED APPLICATIONS

This application claims priority to and benefit of U.S. Ser. No. 61/018,286, filed on Dec. 31, 2007, and U.S. Ser. No. 61/022,115, filed on Jan. 18, 2008, both of which are incorporated herein by reference in their entirety for all purposes.

### STATEMENT AS TO RIGHTS TO INVENTIONS MADE UNDER FEDERALLY SPONSORED RESEARCH AND DEVELOPMENT

This work was supported in part by Grant No: DE-AC02-05CH11231 from the U.S. Department of Energy. The Government of the United States of America has certain rights in this invention.

### FIELD OF THE INVENTION

This invention pertains to the field of diagnostic and screening devices. In particular, in certain embodiments, this invention provides a kinase and/or phosphatase detection system comprising one or more kinase and/or phosphatase substrates attached to a surface comprising nanoscale features that enhance a Raman spectroscopic signal.

### BACKGROUND OF THE INVENTION

The addition to, or removal of, phosphate groups to proteins is important for the transmission of signals within eukaryotic cells and, as a result, protein phosphorylation and dephosphorylation regulate many diverse cellular processes. Normal cell growth is characterized by tightly regulated signal transduction pathways consisting of complex sets of coordinated intracellular signals that modulate or alter cell activity (e.g., growth, proliferation, apoptosis, etc.). In contrast, neoplasms are characterized by deregulated cell growth. In addition, malignant neoplasms have the ability to invade normal tissue as well as metastasize to, and grow at body sites distant from the original neoplasm. The etiology of deregulated cell growth observed in many cancer cells is believed to involve aberrant changes in signaling pathways controlling cellular growth, division, differentiation and apoptosis.

Protein kinases and/or phosphatases have emerged as important cellular regulatory proteins in many aspects of neoplasia. Genetic mutations in protein kinase/phosphatase-mediated signaling processes frequently occur in the initiating events that result in disruption of the normal cell signaling pathways. Protein kinases are enzymes that covalently attach a phosphate group to the side chain typically of tyrosine, serine, or threonine residues found in proteins, while phosphatases are enzymes that remove such phosphate groups. Phosphorylation changes the activity of important signaling proteins. By controlling the activity of these proteins, kinases and/or phosphatases control most cellular processes including, but not limited to, metabolism, transcription, cell cycle progression, cytoskeletal rearrangement, cell movement, apoptosis and differentiation.

With the completion of the human genome sequence, it is estimated that there are approximately 500 protein kinases encoded within the genome (Manning et al. (2002) Science 298: 19 12-1934; Daucey and Sausville (2003) Nature Rev. Drug Discov. 2: 296-313). This represents approximately 1.7% of all human genes (Manning et al. (2002) Science 298: 19 12-1934). Most of the 30 known tumor suppressor genes and more than 100 dominant oncogenes are protein kinases (Futreal et al. (2001) Nature 409: 850-852). Somatic mutations in this group of genes play a role in a significant number of human cancers. Therefore, protein kinases offer an abundant source of potential drug targets at which to intervene in cancer.

While the kinase/phosphatase protein families represent a rich source of new drug targets, developing assays used to determine compound affinity is highly problematic. Current high throughput screening assays for protein kinase and/or phosphatase modulators (e.g., inhibitors or agonists) measure the incorporation into, or loss of, a phosphate from a protein or peptide substrate. The most established method for assaying protein kinase/phosphatase modulators is a radiometric assay in which the gamma phosphate of ATP is labeled with either ³²P or ³³P. When the kinase transfers the gamma phosphate to the hydroxyl of the protein substrate during the phosphor-transferase reaction the protein becomes covalently labeled with the isotope. Conversely, where a phosphatase removes a labeled phosphate, the protein loses the isotopic label. The protein is removed from the labeled ATP and the amount of radioactive protein is determined. Adaptation of this assay into a high throughput format is problematic due to the labor intensive separation steps and the large amounts of radioactivity that are used.

An alternative radiometric assay that is capable of higher throughput is the SPA or scintillation proximity assay. In this assay beads impregnated with a scintillator emit light when the labeled substrate is bound to the bead. This assay is limited by the level of radioactivity and the efficiency of the peptide substrate.

Most non-radioactive assays use antibodies that recognize the product of the kinase reaction, i.e. a phosphopeptide. The binding assays use antibodies detected with enzyme-catalyzed luminescent readout. These methods are limited by reagent availability, well coating, and multiple wash and incubation steps.

### SUMMARY OF THE INVENTION

In certain embodiments this invention pertains to a screening system for the detection and/or quantification of the presence and/or activity of one or more kinases and/or phosphatases in a sample. In certain embodiments, the system comprises a kinase and/or phosphatase substrate with high specificity for a target kinase and/or phosphatase attached to a substrate that enhances a signal in Raman spectroscopy. In certain embodiments, the substrate is a substrate comprising nanoscale features that enhance a signal in a SERs measurement.

Accordingly, in certain embodiments, a device is provided for the detection of kinase and/or phosphatase activity. The device typically comprises a Raman active surface comprising features that enhance Raman scattering where the surface has attached thereto at lease one kinase and/or phosphatase substrate molecule. In certain embodiments the surface has attached thereto a plurality of kinase substrate (e.g., a small molecule, a lipid, a peptide, etc.) and/or phosphatase substrate molecules (e.g., a phosphorylated small molecule, a phosphorylated lipid, a phosphorylated peptide etc.). In certain embodiments the kinase substrate molecules include, but are not limited to nucleotides, sugars, polysaccharides, polymers, and lipids, while the phosphatase substrate molecules include but are not limited to phosphorylated nucleotides, phosphorylated sugars, phosphorylated polysaccharides, phosphorylated polymers, and phosphorylated lipids. In certain embodiments the kinase substrates include peptide substrates for a serine kinase, a threonine kinase, a histidine kinase, and/or a tyrosine kinase. In certain embodiments the substrates are peptide substrates for Src tyrosine kinases. In various embodiments the plurality of peptides comprises at least 3, preferably at least about 5, more preferably at least about 10, 100, 500, 1,000, 2,000, or 5,000 different peptides. In certain embodiments the length of the peptides ranges from about 5 to about 50 amino acids. In certain embodiments the peptides can be localized such that signals from each species of peptide are distinguishable from signals from the other species of peptide. In various embodiments the features that enhance Raman scattering comprise a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers. In certain embodiments the features that enhance Raman scattering comprise a material selected from the group consisting of a metal, a carbon-based material, a polymer, a quartz material, a liquid crystal material, a metal oxide material, a salt crystal, and a semiconductor material. In certain embodiments the features that enhance Raman scattering comprise a material selected from the group consisting of a noble metal, a noble metal alloy, a noble metal composite. In certain embodiments features that enhance Raman scattering comprise a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs. In certain embodiments the center to center distance of the features ranges from about 25 nm, 50 nm, or 50 nm to about 0.5 μm, 300 nm, 200 nm, or 150 nm. In certain embodiments the features that enhance Raman scattering have a size that ranges from about 10 nm, 15 nm, 20 nm, 25 nm, 30 nm, 40 nm, or 50 nm to about 200 nm, 150 nm, 100 nm, or 75 nm. In various embodiments the surface has attached thereto a plurality of kinase and/or phosphatase substrate molecules (e.g., at least 2, preferably at least 5, or 10, more preferably at least 20, 50, or 100, different species). In certain embodiments the device comprises a Raman active surface comprising gold nanopyramids; the kinase substrate molecule comprises a plurality of protein kinase and/or phosphatase substrates; and the Raman active surface comprises or is disposed within a microfluidic chamber.

Also provided are methods of detecting and/or quantifying kinase or phosphatase activity in a sample. The methods typically involve contacting the sample with a molecule comprising a kinase and/or phosphatase substrate sequence; and detecting phosphorylation of the molecule by detecting a change in the Raman scattering spectrum of the peptide. In certain embodiments the surface has attached thereto a plurality of kinase substrate (e.g., a small molecule, a lipid, a peptide, etc.) and/or phosphatase substrate molecules (e.g., a phosphorylated small molecule, a phosphorylated lipid, a phosphorylated peptide etc.). In certain embodiments the kinase substrate molecules include, but are not limited to nucleotides, sugars, polysaccharides, polymers, and lipids, while the phosphatase substrate molecules include but are not limited to phosphorylated nucleotides, phosphorylated sugars, phosphorylated polysaccharides, phosphorylated polymers, and phosphorylated lipids. In certain embodiments the kinase substrates include peptide substrates for a serine kinase, a threonine kinase, a histidine kinase, and/or a tyrosine kinase. In certain embodiments the substrates are peptide substrates for Src tyrosine kinases. In various embodiments the plurality of peptides comprises at least 3, preferably at least about 5, more preferably at least about 10, 100, 500, 1,000, 2,000, or 5,000 different peptides. In certain embodiments the length of the peptides ranges from about 5 to about 50 amino acids. In certain embodiments the peptides can be localized such that signals from each species of peptide are distinguishable from signals from the other species of peptide. In various embodiments the features that enhance Raman scattering comprise a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers. In certain embodiments the features that enhance Raman scattering comprise a material selected from the group consisting of a metal, a carbon-based material, a polymer, a quartz material, a liquid crystal material, a metal oxide material, a salt crystal, and a semiconductor material. In certain embodiments the features that enhance Raman scattering comprise a material selected from the group consisting of a noble metal, a noble metal alloy, a noble metal composite. In certain embodiments features that enhance Raman scattering comprise a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs. In certain embodiments the center to center distance of the features ranges from about 25 nm, 50 nm, or 50 nm to about 0.5 μm, 300 nm, 200 nm, or 150 nm. In certain embodiments the features that enhance Raman scattering have a size that ranges from about 10 nm, 15 nm, 20 nm, 25 nm, 30 nm, 40 nm, or 50 nm to about 200 nm, 150 nm, 100 nm, or 75 nm. In various embodiments the surface has attached thereto a plurality of kinase and/or phosphatase substrate molecules (e.g., at least 2, preferably at least 5, or 10, more preferably at least 20, 50, or 100, different species). In certain embodiments the device comprises a Raman active surface comprising gold nanopyramids; the kinase substrate molecule comprises a plurality of protein kinase and/or phosphatase substrates; and the Raman active surface comprises or is disposed within a microfluidic chamber.

Also provided are systems for the detection of kinase and/or phosphatase activity in one or more samples. In various embodiments the systems comprise a device for the detection of kinase and/or phosphatase activity as described herein (e.g., a Raman active surface comprising features that enhance Raman scattering where the surface has attached thereto at lease one kinase and/or phosphatase substrate molecule); and a Raman detection probe disposed to measure surface enhanced Raman spectra from one or more regions of the device. In certain embodiments the device and/or the Raman detection probe are disposed in a positioner. In certain embodiments the device is disposed on an x-y scanning sample stage. In certain embodiments the Raman detection probe comprises a laser light delivery fiber, an objective lens, a long-pass optical filter, and a Raman scattering light collection fiber. In various embodiments the system can further comprise; a control computer that controls data acquisition location.

In certain embodiments methods are provided for screening a sample for a modulator of kinase and/or phosphatase activity. The methods typically involve contacting a device for the detection of kinase and/or phosphatase activity as described herein (e.g., a Raman active surface comprising features that enhance Raman scattering where the surface has attached thereto at lease one kinase and/or phosphatase substrate molecule); performing a SERS measurement to detect a change in the Raman scattering spectrum when the kinase and/or phosphatase substrates are phosphorylated or dephosphorylated, where an inhibition in change of the Raman spectrum indicates that a test agent is an inhibitor of kinase and/or phosphatase activity. In certain embodiments the test sample comprises a library of test agents. In certain embodiments the test sample comprises a library of test agents comprising at least 10 or at least 20, preferably at least 50 or at least 100, more preferably at least 200, 300, 400, or 500, and most preferably at least 1,000, or at least 5,000 different test agents.

In various embodiments method of making a surface for detection of kinase and/or phosphatase activity are provided, the method comprising depositing an array of kinase and/or phosphatase substrate molecules on a first surface; contacting the array of kinase and/or phosphatase substrate molecules with a SERS surface comprising a plurality of features that enhance Raman scattering, where the contacting is under conditions that transfer the kinase and/or phosphatase substrate molecules from the first surface to the SERS surface to form a surface for the detection of kinase and/or phosphatase activity. In certain embodiments the kinase and/or phosphatase substrate molecules bear a functional group or a linker having a functional group that reacts to form a covalent linkage with the SERS surface. In certain embodiments the SERS surface is formed on a soft-lithographic substrate (e.g., a PDMS chip). In certain embodiments the method further comprises disposing the SERs surface in or attaching the SERs surface to a microfluidic structure to form a well adjacent to the SERS surface. In certain embodiments the well has a volume of 1 μL or less, or 0.5 μL or less, or 0.25 μL or less. In certain embodiments the array of kinase and/or phosphatase substrate molecules comprises a spacing between dots that ranges from about 20 to about 500 nm. In certain embodiments the dots forming the array of kinase and/or phosphatase substrate molecules have a characteristic dimension that ranges from about 20 to about 500 nm. In certain embodiments the array comprises at least at least 3, preferably at least about 5, more preferably at least about 10, 20, 30, 40, or 50, and most preferably at least 100, 500, 1,000, 2,000, or 5,000 different substrates. In certain embodiments the kinase substrate molecules are selected from the group consisting of a small molecule, a lipid, and a peptide. In certain embodiments the phosphatase substrate molecules are selected from the group consisting of a phosphorylated small molecule, a phosphorylated lipid, and a phosphorylated peptide. In certain embodiments the kinase substrate molecules are selected from the group consisting of nucleotides, sugars, polysaccharides, polymers, and lipids. In certain embodiments the phosphatase substrate molecules are selected from the group consisting of phosphorylated nucleotides, phosphorylated sugars, phosphorylated polysaccharides, phosphorylated polymers, and phosphorylated lipids. In certain embodiments the kinase and/or phosphatase substrate molecules are peptides. In certain embodiments the kinase substrates include peptide substrates for a serine kinase, a threonine kinase, a histidine kinase, and/or a tyrosine kinase. In certain embodiments the substrates are peptide substrates for Src tyrosine kinases. In certain embodiments the length of the peptides ranges from about 5 to about 50 amino acids. In certain embodiments the peptides can be localized such that signals from each species of peptide are distinguishable from signals from the other species of peptide. In various embodiments the features that enhance Raman scattering comprise a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers. In certain embodiments the features that enhance Raman scattering comprise a material selected from the group consisting of a metal, a carbon-based material, a polymer, a quartz material, a liquid crystal material, a metal oxide material, a salt crystal, and a semiconductor material. In certain embodiments the features that enhance Raman scattering comprise a material selected from the group consisting of a noble metal, a noble metal alloy, a noble metal composite. In certain embodiments features that enhance Raman scattering comprise a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs. In certain embodiments the center to center distance of the features ranges from about 25 nm, 50 nm, or 50 nm to about 0.5 μm, 300 nm, 200 nm, or 150 nm. In certain embodiments the features that enhance Raman scattering have a size that ranges from about 10 nm, 15 nm, 20 nm, 25 nm, 30 nm, 40 nm, or 50 nm to about 200 nm, 150 nm, 100 nm, or 75 nm.

Methods are also provided for fabricating a nanopyramid surface. The methods typically involve providing a photolithographable surface; contacting the surface with a first plasma to produce a nanoscale oxide island array; etching the surface to form a nanopillar array; removing the oxide layer on the nanopillars comprising the nanopillar array; and etching the nanopillar array to form a nanopyramid array. In certain embodiments the method further comprises metalizing the nanopyramid array. In certain embodiments the photolithographable surface comprises a silicon or germanium surface. In certain embodiments the photolithographable surface comprises a material selected from the group consisting of ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. In certain embodiments the first plasma comprises a mixture of HBr and O₂. In certain embodiments the etching the surface to form a nanopillar array comprises etching by HBr plasma. In certain embodiments the oxide island layer is removed by SF₆ plasma etching. In certain embodiments the etching the nanopillar array to form a nanopyramid array comprises etching by HBr plasma. In certain embodiments the metalizing comprises depositing a layer of metal on the nanopyramid array where the metal comprises a metal selected from the group consisting of a noble metal, a noble metal alloy, and a noble metal composite. In certain embodiments the metalizing comprises depositing a layer of metal on the nanopyramid array where the metal comprises a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs.

Also provided are nanopyramid arrays. The nanopyramid arrays can be used in the creation of Raman-active surfaces. In various embodiments the array comprises a surface having thereon a plurality of nanopyramids where the nanopyramids have a characteristic dimension averaging less than about 100 nm, and an average interfeature spacing comprising less than about 500 nm. In various embodiments the nanopyramids have a characteristic dimension averaging less than about 50 nm, and an average interfeature spacing comprising less than about 100 nm. In certain embodiments the surface comprises a metal selected from the group consisting of a noble metal, a noble metal alloy, and a noble metal composite. In certain embodiments the surface comprises a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs.

### DEFINITIONS

A “kinase” is a molecule that catalyzes the transfer of a phosphate group (e.g., from ATP or other molecule) to a target molecule such a peptide or other kinase substrate.

A “kinase substrate” refers to a molecule that can be phosphorylated or, in certain instances, dephosphorylated by a kinase.

A “phosphatase” is a molecule that catalyzes the transfer of a phosphate group from a target molecule such a peptide or other phosphatase substrate thereby resulting in the partial or complete dephosphorylation of that substrate.

A “phosphatase substrate” refers to a molecule that can be partial or fully dephosphorylated by a phosphatase.

An “array” refers to a collection of different species of molecule on a solid support (e.g., a surface). In certain embodiments different species of molecule are located at different regions of the support, i.e., they are spatially addressed.

The term “array feature” refers to a substantially contiguous domain of an array that predominantly comprises a single species of molecule (e.g. a spot on an array).

A “Raman-active substrate” refers to a substrate suitable for Surface Enhances Raman Spectroscopy (SERs). In certain embodiments the Raman-active substrate comprises nanoscale features that enhance a Raman scattering signal.

The term “plurality of kinase substrates” when used in reference to a kinase substrate array indicates that the array contains at least two different kinase substrates (e.g., different peptides) at different locations. Similar a “plurality of phosphatase substrates” indicates that the array contains at least two different phosphatase substrates (e.g., different peptides) at different locations. In certain embodiments a substrate can be both a kinase and phosphatase substrate.

### BRIEF DESCRIPTION OF THE DRAWINGS

FIGS. 1A-1D illustrate a detection scheme for protein phosphorylation (e.g., tyrosine phosphorylation) by a kinase (e.g., SRC). FIG. 1A: Illustrates a substrate peptide for Src kinase (SEQ ID NO:1). It has 10 amino acids including cysteine residue at N-terminal and Tyrosine residue near C-terminal. FIG. 1B: Phosphorylation of substrate peptide. The hydroxyl group on the tyrosine residue is substituted by a negatively charged phosphate group after peptide phosphorylation by Src kinase. FIG. 1C: Phosphor-peptide folding on enhanced scattering Au surface. The negative charges of phosphor-tyrosine residue is attracted closer to the electrophilic Au surface where the Raman scattering enhancement is stronger. FIG. 1D: Calculated relative SERS enhancement factor vs the distance of Tyrosine residue to Au surface. The closer the Tyrosine residue is to the Au surface, the stronger the SERS signal can be obtained and vise versa.

FIGS. 2A-2D shows SERs spectral of tyrosine phosphorylation of tyrosine by Src kinase. FIG. 2A shows SERs spectral of the phosphorylated and unphorphorylated substrate. FIG. 2B shows real time Tyrosine phosphorylation by purified Src kinase. The 1004 cm⁻¹ is attributed to the phenyl ring breathing mode of tyrosine residue. With more and more peptide phosphorylation, the tyrosine Raman peak becomes stronger and stronger indicating the peptide folding due to the negatively charges of phosphate group. FIG. 2C shows time-resolved phosphorylation level or the 1004 cm⁻¹ peak enhancement. The detection limit is 10 pM Src kinase in 10 μL volume, which renders sub-femtomole kinase detection sensitivity. FIG. 2C shows a real-time inhibition experiment. The graph shows phosphorylation level versus the inhibitor concentration for single site blocking and double site block inhibitor enzymes. The IC 50 concentrations for these two inhibitors are 44 nM and 20 nM, respectively.

FIGS. 3A-3D show inhibition drug screening data. FIG. 3A shows final spectra after 20 min reaction with 10 nM Src kinase and Src inhibitor I in different concentrations. FIG. 3B shows time-lapse phosphorylation level in the reactions with 10 nM Src kinase and Src inhibitor I. FIG. 3C shows finial phosphorylation level after 20 min reaction for various inhibitor concentrations. The IC50 concentration of Src inhibitor I is around 50 nM after sigmoid fitting of the data. FIG. 3 D shows time-lapse phosphorylation level in the reactions with 10 nM Src kinase and AMP-PNP without ATP.

FIGS. 4A-4D show real time tyrosine phosphorylation detection in crude cell lysate. FIG. 4A shows a real time SERS spectrum of peptide after introducing crude lysate of wild-type 3T9 mouse fibroblast cells. FIG. 4B shows a real time SERS spectra of peptide after introducing crude lysate of 3T9 mouse fibroblast cells transfected with virus Src protein. FIG. 4C shows the time-resolved phosphorylation level. The tyrosine phosphorylation level is dramatically elevated in Src+ cells. FIG. 4D shows the phosphorylation level of different type of 3T9 cells.

FIG. 5, panels A-E show inhibition drug screening data.

FIG. 6 illustrates a nanopyramid array.

FIG. 7 illustrates a protocol for fabricating a nanopyramid array.

FIG. 8 schematically illustrates a protocol for fabricating a surface comprising a plurality of nanoscale features.

FIG. 9 schematically illustrates a method of fabricating a SERs kinase detection substrate.

FIG. 10 schematically illustrates a SERS detection system for detecting kinase activity on a nanopyramid substrate.

FIG. 11 schematically illustrates one configuration of an automated SERs detection system.

### DETAILED DESCRIPTION

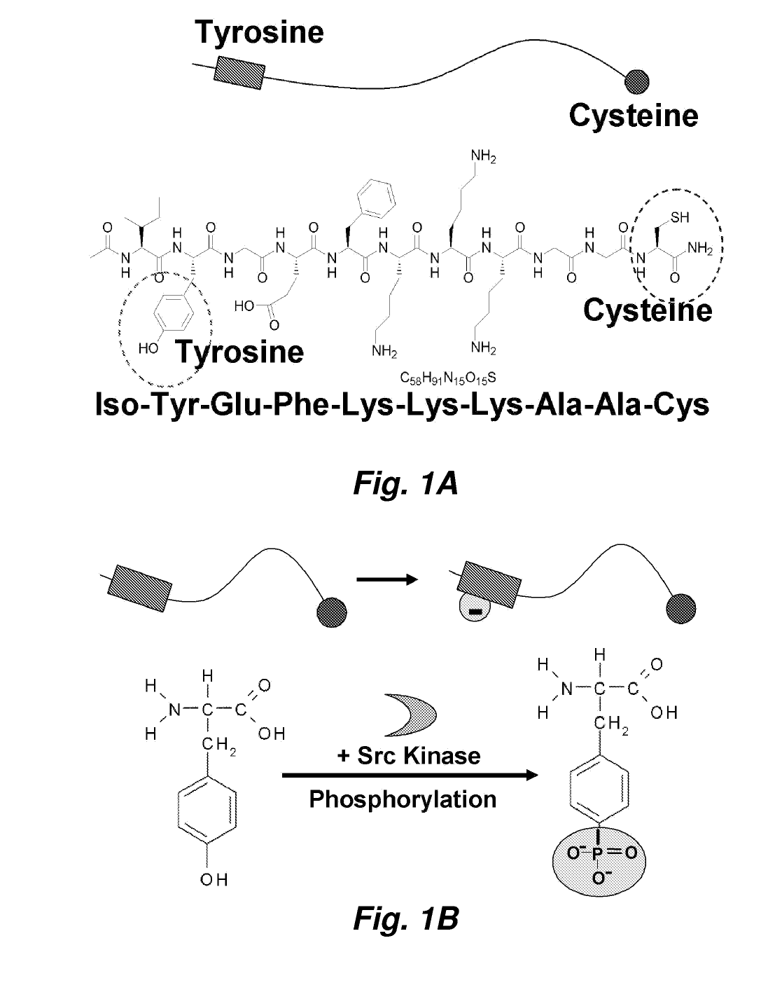

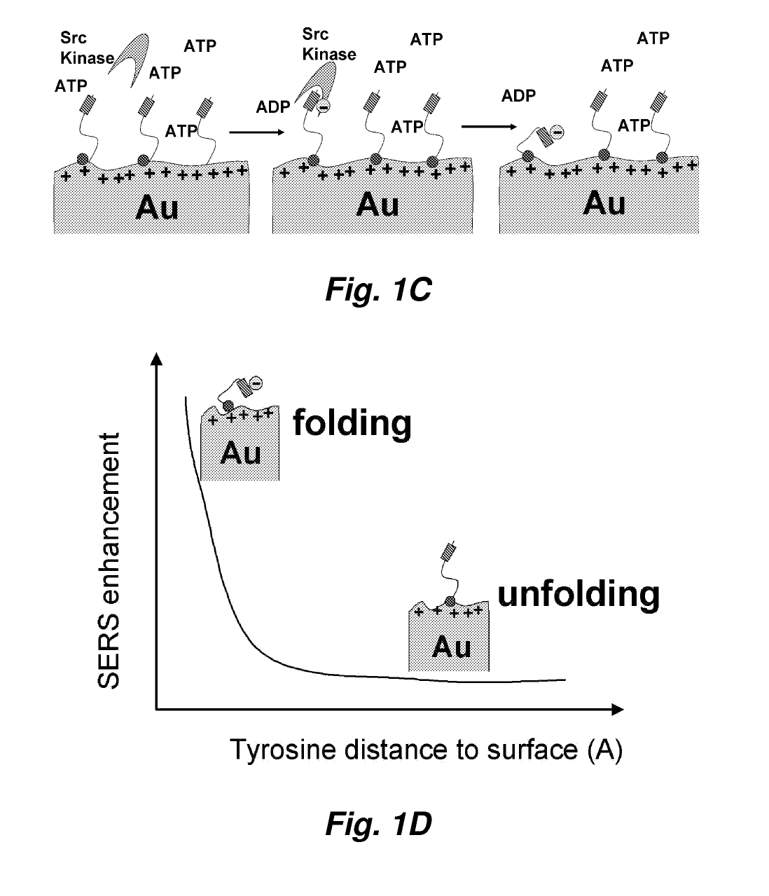

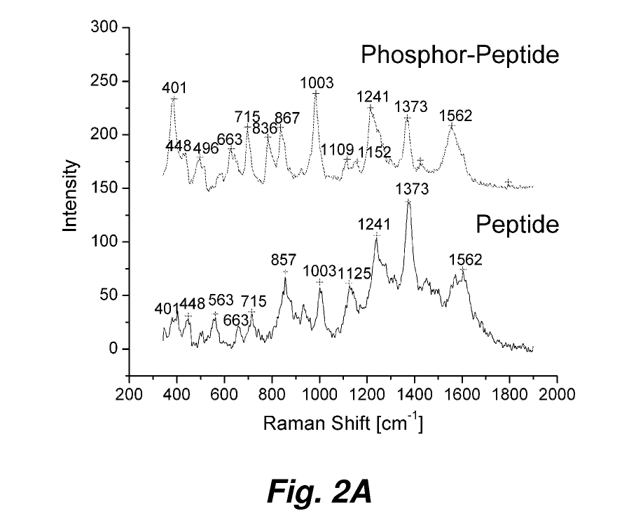

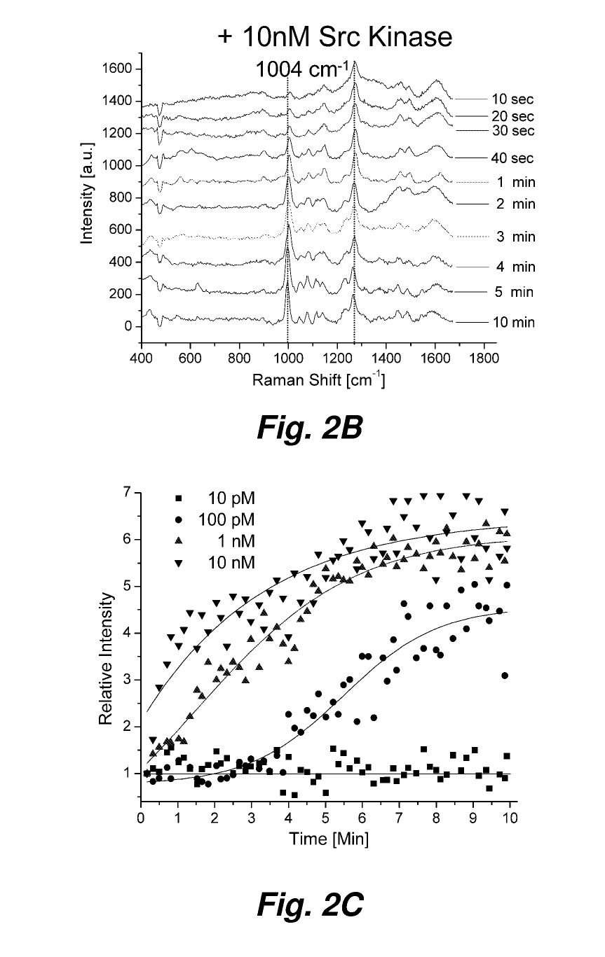

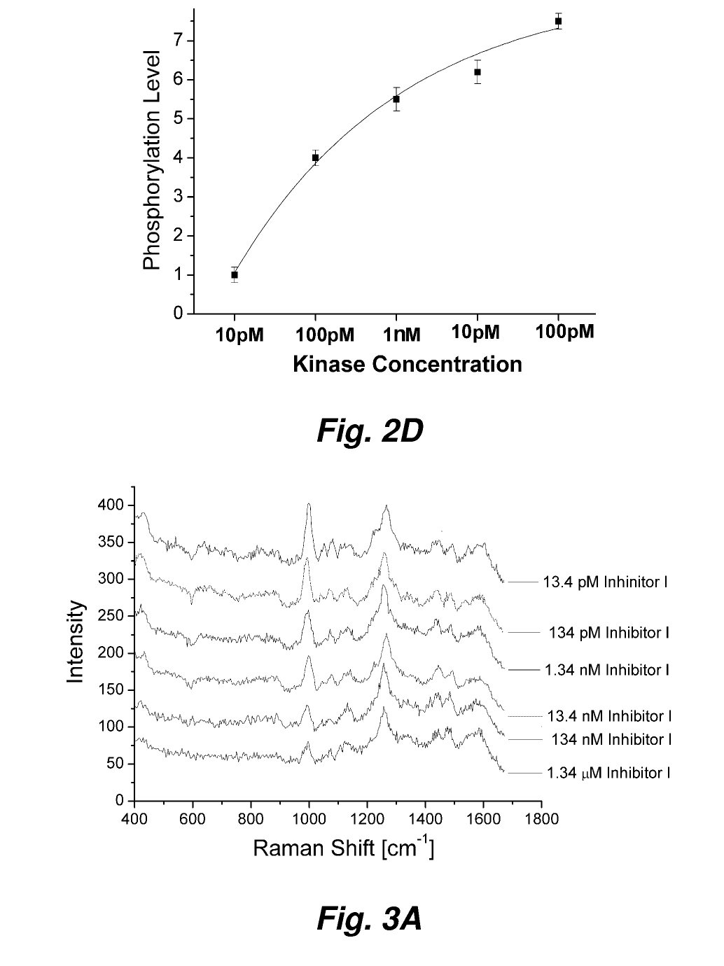

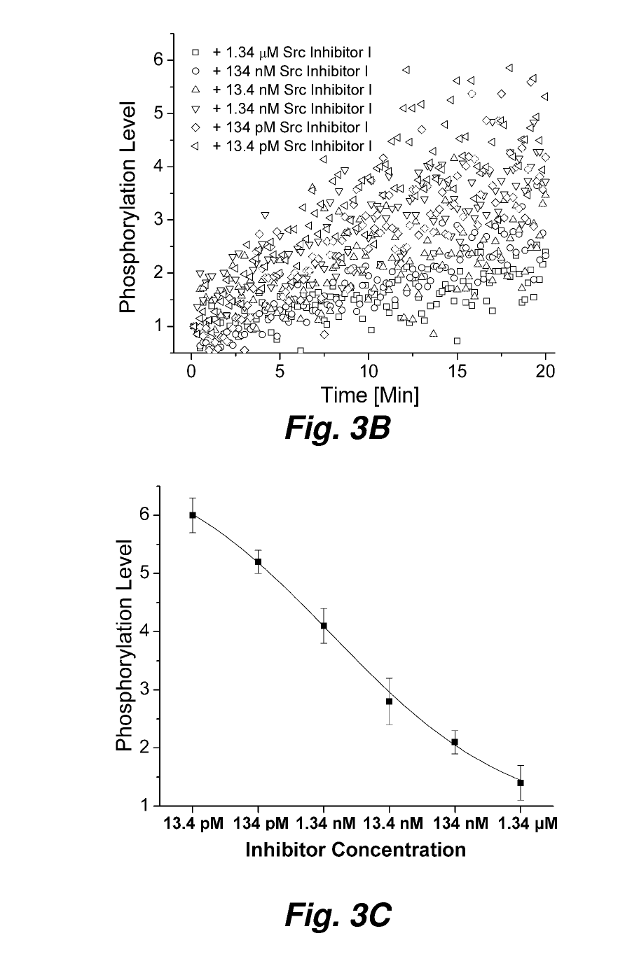

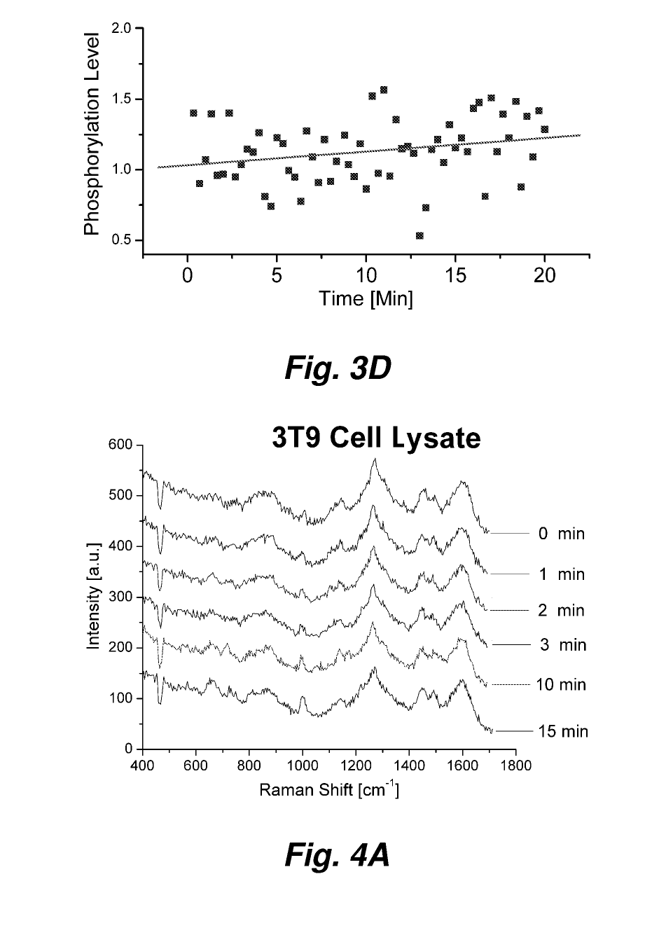

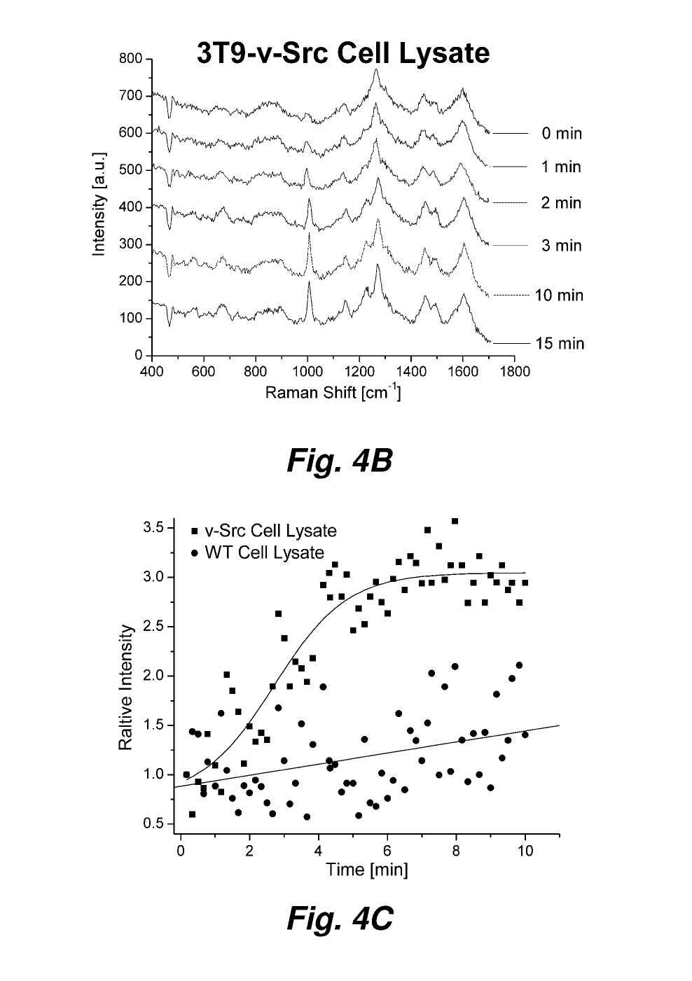

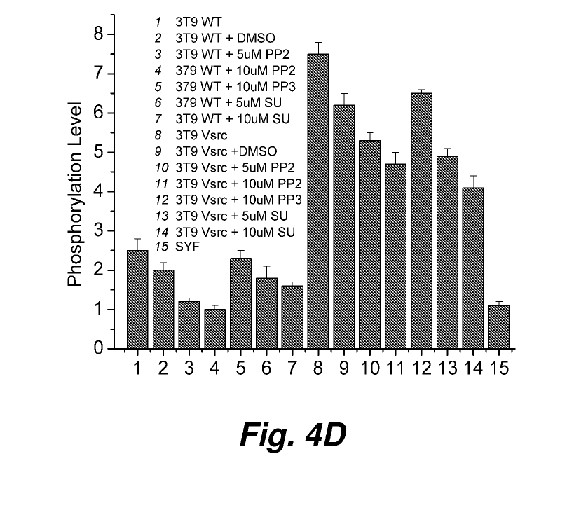

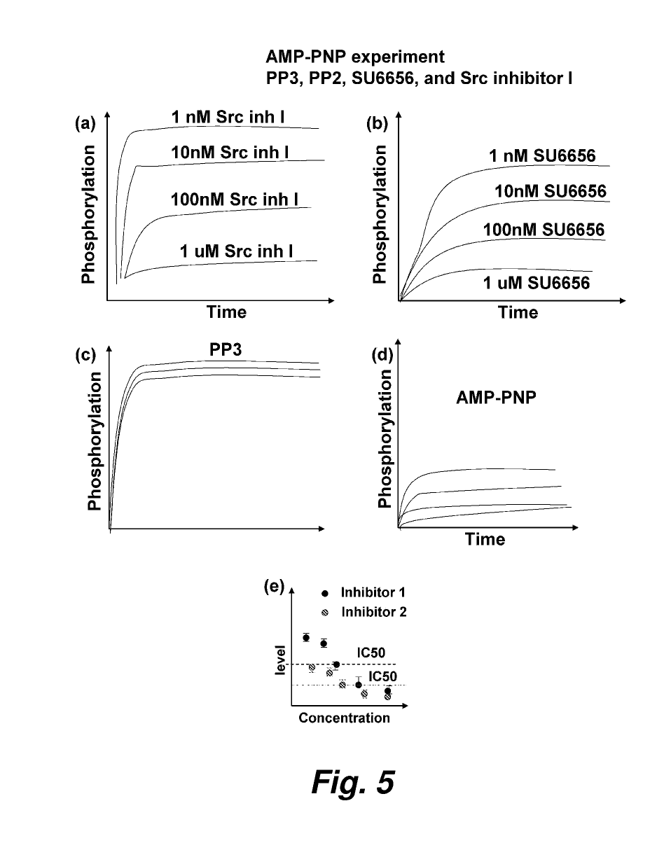

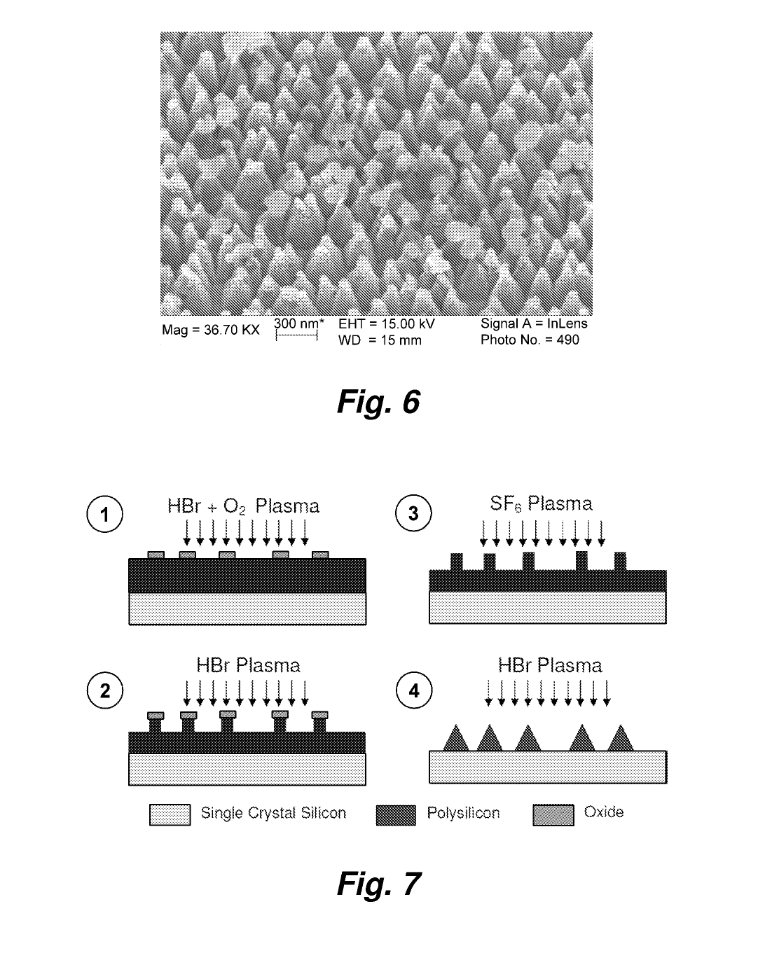

This invention provides novel compositions and methods for investigating/characterizing the interactions of protein/peptide substrates and kinases (enzymes that phosphorylate a protein) and/or phosphatases (enzymes that partially or fully dephosphorylate a protein). It was a surprising discovery that under appropriate conditions, Raman spectroscopy can be used to effectively detect and/or quantitate the phosphorylation (or dephosphorylation) of a target molecule, e.g., a kinase and/or phosphatase substrate (see, e.g., FIGS. 1A and 1B). An illustrative Raman spectra of a peptide before and after phosphorylation reaction is shown in FIG. 2A. As shown in this figure, phosphorylation of the kinase substrate (in this case a peptide) causes the intensity of several Raman peaks to change, some peaks to shift and new peaks to appear. Clearly, phosphorylation of the kinase substrate can be detected using Raman spectroscopy. Similarly, dephosphorylation of a phosphorylated substrate can also be detected using Raman spectroscopy. Accordingly, in certain embodiments, this invention provides a detection composition and/or detection system comprising one or more kinase and/or phosphatase substrate molecule(s) e.g., molecule(s) that can be phosphorylated by a kinase and/or dephosphorylated by a phosphatase) attached (e.g. chemically conjugated) to one or more nanoparticle(s), or more preferably to a nanoscale feature or features(s) on a surface where the nanoparticle or nanoscale feature or features act to enhance the signal produced in Raman spectroscopy.

In various embodiments, particularly where the kinase and/or phosphatase substrate is attached to a nanoscale feature or features on a surface (e.g., a Raman active surface), different kinase and/or phosphatase substrate molecules can be localized at different positions on the substrate thereby forming an array for the detection of one or more kinases and/or phosphatases and/or the quantitation of the activity of one or more kinases and/or phosphatases.

FIG. 1C illustrates the phosphorylation of a kinase substrate (e.g., a peptide) on a Raman active surface, e.g., an enhanced scattering gold surface. The negative charge of the phosphorylated residue (in this case tyrosine) is attracted closer to the electrophilic gold surface where the Raman scattering enhancement is stronger thereby improving the Raman signal. As shown in FIG. 1D the closer the phosphorylated residue is to the gold surface, the stronger the SERS signal can be obtained and vise versa (e.g., for detection of dephosphorylation).

Surfaces, particularly Raman active surfaces comprising an array of attached kinase and/or phosphatase substrates thus provide an effective tool for real-time screening for the presence and/or activity of one or a plurality of kinases and/or phosphatases and/or for quantification of the kinetics of one or more kinases and/or phosphatases. The kinase and/or phosphatase substrate arrays can also be readily used to screen for kinase and/or phosphatase inhibitor (or agonistic) activity of one or a plurality of test agents (e.g. a chemical library).

Thus in various embodiments the kinase/phosphatase screening systems described herein can be used for rapid and effective screening of kinase and/or phosphatase inhibitor drugs (more than 50% cancer drugs are kinase inhibitor drugs) or agents that upregulate kinase and/or phosphatase activity. In certain embodiments the kinase/phosphatase assays can also be used for example in personalized molecular diagnostics for cancers by physicians and hospital personnel.

Raman-active surfaces, e.g., surfaces comprising nanoscale features that enhance a Raman spectroscopy signal are particularly well suited for use as array surfaces in the SERs kinase substrate arrays described herein. While a variety of nanoscale features (e.g., nanorods, nanopillars, nanowires, nanotubes, nanodiscs, nanocrescents, nanoscale dots, nanoscale fibers, nanotubes, nanohorns, nanoholes, nano bowties, nanobowls, nanocrescents, and nanoburgers, and other nanoplasmonically enhanced nanostructures) are well suited for enhancement of Raman signals, in certain embodiments, arrays of nanopyramids are used as the array surface. In certain embodiments, this invention also provides for a batch fabrication method to make nanoscale pyramid arrays (see, e.g., FIG. 7). The process procedure is compatible with conventional integrated circuit fabrication process, so the nanopyramid arrays can be made with very high yield and large quantities at once. In addition, the array can be patterned using optical lithography. The nanoscale gold pyramid array contains large numbers of sub-10 nm gaps between adjacent pyramids and the electromagnetic intercoupling across the small gaps create very high local field enhancement and the Raman scattering signal can be enhanced for tens orders of magnitude. Other engineered SERS nanostructures such as nanodots can also be patterned into an SERS microarray.

It will also be recognized that while the various methods and compositions are described with respect to detecting phosphorylation of a substrate, they can also be sued to detect dephosphorylation of a substrate and/or the degree of phosphorylation of a substrate.

### I. Kinase/Phosphatase Substrates for Use in SERS Kinase/Phosphatase Assays.

Essentially any molecule that can be phosphorylated by a kinase and/or dephosphorylated by a phosphatase can be used as a kinase/phosphatase substrate in the methods and compositions described herein. While proteins/peptides comprise the largest substrate class for kinases and phosphatases, a number of other kinase and/or phosphatase substrates are known as well. Such substrates include, but are not limited to various sugars (e.g., hexose, glucose, fructose, mannose, etc.), nucleotides/nucleic acids, acetate, butyrate, fatty acids, sphinganine, diacylglycerol, ceramide, and the like. By way of illustration, a number of kinases, and their Enzyme Commission numbers (EC numbers), and by implication, kinase substrates are shown in Table 1. It will be recognized that for most, if not all kinase substrates there exists a corresponding phosphatase.

TABLE 1
Illustrative kinases and corresponding Enzyme Commission
number (E.C. number).

| E.C. | | E.C. | |
|---|---|---|---|
| No. | Kinase | No. | Kinase |
| 2.7.1.32 | Choline kinase | 2.7.1.90 | Diphosphate - fructose-6- |
| | | | phosphate 1- |
| | | | phosphotransferase |
| 2.7.1.37 | Protein kinase 2.7.1.38 | 2.7.1.91 | Sphinganine kinase |
| | Phosphorylase kinase | | |
| 2.7.1.39 | Homoserine kinase | 2.7.1.107 | Diacylglycerol kinase |
| 2.7.1.67 | 1-phosphatidylinositol 4-kinase | 2.7.1.138 | Ceramide kinase |
| 2.7.1.72 | Streptomycin 6-kinase | 2.7.1.2 | Glucokinase |
| 2.7.1.82 | Ethanolamine kinase | 2.7.1.3 | Ketohexokinase |
| 2.7.1.87 | Streptomycin 3″-kinase | 2.7.1.4 | Fructokinase |
| 2.7.1.95 | Kanamycin kinase | 2.7.1.11 | 6-phosphofructokinase |
| 2.7.1.100 | 5-methylthioribose kinase | 2.7.1.15 | Ribokinase |
| 2.7.1.103 | Viomycin kinase | 2.7.1.20 | Adenosine kinase |
| 2.7.1.109 | [Hydroxymethylglutaryl-CoA | 2.7.1.35 | Pyridoxal kinase |
| | reductase (NADPH2)] kinase | | |
| 2.7.1.112 | Protein-tyrosine kinase | 2.7.1.45 | 2-dehydro-3- |
| | | | deoxygluconokinase |
| 2.7.1.116 | [Isocitrate dehydrogenase | 2.7.1.49 | Hydroxymethylpyrimidine |
| | (NADP+)] kinase | | kinase |
| 2.7.1.117 | [Myosin light-chain] kinase | 2.7.1.50 | Hydroxyethylthiazole kinase |
| 2.7.1.119 | Hygromycin-B kinase | 2.7.1.56 | 1-phosphofructokinase |
| 2.7.1.123 | Calcium/calmodulin dependent | 2.7.1.73 | Inosine kinase |
| | Protein kinase | | |
| 2.7.1.125 | Rhodopsin kinase | 2.7.1.92 | 5-dehydro-2- |
| | | | deoxygluconokinase |
| 2.7.1.126 | [Beta-ad renergic-receptor] | 2.7.1.144 | Tagatose-6-phosphate kinase |
| | kinase | | |
| 2.7.1.129 | [Myosin heavy-chain] kinase | 2.7.1.146 | ADP-dependent |
| | | | phosphofructokinase |
| 2.7.1.135 | [Tau protein] kinase | 2.7.1.147 | ADP-dependent glucokinase |
| 2.7.1.136 | Macrolide 2′-kinase | 2.7.4.7 | Phosphomethylpyrimidine |
| | | | kinase |
| 2.7.1.137 | 1-phosphatidylinositol 3-kinase | 2.7.6.2 | Thiamin pyrophosphokinase |
| 2.7.1.141 | [RNA-polymerase]-subunit | 2.7.1.31 | Glycerate kinase |
| | kinase | | |
| 2.7.1.153 | Phosphatidylinositol-4,5- | 2.7.4.6 | Nucleoside-diphosphate kinase |
| | bisphosphate 3-kinase | | |
| 2.7.1.154 | Phosphatidylinositol-4- | 2.7.6.3 | 2-amino-4-hydroxy-6- |
| | phosphate 3-kinase | | hydroxymethyldihydropteridine |
| | | | pyrophosphokinase |
| 2.7.1.68 | 1-phosphatidylinositol-4- | 2.7.3.1 | Guanidoacetate kinase |
| | phosphate 5-kinase | | |
| 2.7.1.127 | 1D-myo-inositol-trisphosphate | 2.7.3.2 | Creatine kinase |
| | 3-kinase | | |
| 2.7.1.140 | Inositol-tetrakisphosphate 5- | 2.7.3.3 | Arginine kinase |
| | kinase | | |
| 2.7.1.149 | 1-phosphatidylinositol 5- | 2.7.3.5 | Lombricine kinase |
| | phosphate 4-kinase | | |
| 2.7.1.150 | 1-phosphatidylinositol 3- | 2.7.1.37 | Protein kinase (Histidine |
| | phosphate 5-kinase | | kinase) |
| 2.7.1.151 | Inositol-polyphosphate | 2.7.1.99 | [Pyruvate |
| | multikinase | | dehydrogenase(lipoamide)] |
| | | | kinase |
| 2.7.4.21 | Inositol-hexakisphosphate | 2.7.1.115 | [3-methyl-2-oxobutanoate |
| | kinase | | dehydrogenase (lipoamide)] |
| | | | kinase |
| 2.7.1.134 | Inositol-tetrakisphosphate 1- | 2.7.1.1 | Hexokinase |
| | kinase | | |
| 2.7.9.1 | Pyruvate, phosphate dikinase | 2.7.1.2 | Glucokinase |
| 2.7.9.2 | Pyruvate, water dikinase | 2.7.1.4 | Fructokinase |
| 2.7.1.12 | Gluconokinase | 2.7.1.5 | Rhamnulokinase |
| 2.7.1.19 | Phosphoribulokinase | 2.7.1.7 | Mannokinase |
| 2.7.1.21 | Thymidine kinase | 2.7.1.12 | Gluconokinase |
| 2.7.1.22 | Ribosylnicotinamide kinase | 2.7.1.16 | L-ribulokinase |
| 2.7.1.24 | Dephospho-CoA kinase | 2.7.1.17 | Xylulokinase |
| 2.7.1.25 | Adenylylsulfate kinase | 2.7.1.27 | Erythritol kinase |
| 2.7.1.33 | Pantothenate kinase | 2.7.1.30 | Glycerol kinase |
| 2.7.1.37 | Protein kinase (bacterial) | 2.7.1.33 | Pantothenate kinase |
| 2.7.1.48 | Uridine kinase | 2.7.1.47 | D-ribulokinase |
| 2.7.1.71 | Shikimate kinase | 2.7.1.51 | L-fuculokinase |
| 2.7.1.74 | Deoxycytidine kinase | 2.7.1.53 | L-xylulokinase |
| 2.7.1.76 | Deoxyadenosine kinase | 2.7.1.55 | Allose kinase |
| 2.7.1.78 | Polynucleotide 5′- | 2.7.1.58 | 2-dehydro-3- |
| | hydroxylkinase | | deoxygalactonokinase |
| 2.7.1.105 | 6-phosphofructo-2-kinase | 2.7.1.59 | N-acetylglucosamine kinase |
| | 2.7.1.113 Deoxyguanosine | | |
| | kinase | | |
| 2.7.1.130 | Tetraacyldisaccharide 4′-kinase | 2.7.1.60 | N-acylmannosamine kinase |
| 2.7.1.145 | Deoxynucleoside kinase | 2.7.1.63 | Polyphosphate-glucose |
| | 2.7.1.156 Adenosylcobinamide | | phosphotransferase |
| | kinase | | |
| 2.7.4.1 | Polyphosphate kinase 2.7.4.2 | 2.7.1.85 | Beta-glucoside kinase |
| | Phosphomevalonate kinase | | |
| 2.7.4.3 | Adenylate kinase | 2.7.2.1 | Acetate kinase |
| 2.7.4.4 | Nucleoside-phosphate kinase | 2.7.2.7 | Butyrate kinase |
| 2.7.4.8 | Guanylate kinase | 2.7.2.14 | Branched-chain-fatty-acid |
| | | | kinase |
| 2.7.4.9 | Thymidylate kinase | 2.7.2. | Propionate kinase |
| 2.7.4.10 | Nucleoside-triphosphate- | 2.7.1.40 | Pyruvate kinase |
| | adenylate kinase | | |
| 2.7.4.13 | (Deoxy)nucleoside-phosphate | .7.1.36 | Mevalonate kinase |
| | kinase | | |
| 2.7.4.14 | Cytidylate kinase | 2.7.1.39 | Homoserine kinase |
| 2.7.4. | Uridylate kinase | 2.7.1.46 | L-arabinokinase |
| 2.7.1.37 | Protein kinase (HPr kinase/ | 2.7.1.52 | Fucokinase |
| | phosphatase) | | |
| 4.1.1.32 | Phosphoenolpyruvate | 2.7.1.71 | Shikimate kinase |
| | carboxykinase (GTP) | | |
| 4.1.1.49 | Phosphoenolpyruvate | 2.7.1.148 | 4-(cytidine 5′-diphospho)-2- |
| | carboxykinase (ATP) | | Cmethyl-D-erythritol kinase |
| 2.7.2.3 | Phosphoglycerate kinase | 2.7.4.2 | Phosphomevalonate kinase |
| 2.7.2.10 | Phosphoglycerate kinase (GTP) | 2.7.4.16 | Thiamine-phosphate kinase |
| 2.7.2.2 | Carbamate kinase | 2.7.9.3 | Selenide, water dikinase |
| 2.7.2.4 | Aspartate kinase | 2.7.1.26 | Riboflavin kinase |
| 2.7.2.8 | Acetylglutamate kinase | 2.7.1.29 | Glycerone kinase |
| 2.7.2.11 | Glutamate 5-kinase | 2.7.1.31 | Glycerate kinase |
| 2.7.1.11 | 6-phosphofructokinase | 2.7.4.1 | Polyphosphate kinase |
| 2.7.1.23 | NAD(+) kinase | 2.7.1.108 | Dolichol kinase |
| 2.7.1.56 | 1-phosphofructokinase | 2.7.1.66 | Undecaprenol kinase |

The substrate and/or substrate consensus sequences are known for a large number of kinases and/or phosphatases. Many of the residues within these consensus sequences have proven to be crucial recognition elements, and the very simplicity of these motifs has made them useful in the study of protein kinases and/or phosphatases and their substrates. Short synthetic oligopeptides based on consensus motifs are typically excellent substrates for protein kinase/phosphatase activity assays. Table 2, below, summarizes some of the known data about specificity motifs for various well-studied protein kinases, along with examples of known phosphorylation sites in specific proteins. A more extensive list can be found in Pearson and Kemp (1991) Meth. Enzymol., 200: 68-82, which is incorporated herein by reference.

TABLE 2
Shows recognition motifs and substrate sequences for some well known kinases.
The phosphoacceptor residue is underlined, amino acids which can function
interchangeably at a particular residue are separated by a slash (/), and
residues that do not appear to contribute strongly to recognition are
indicated by an “X”.

| | Recognition | Phosphorylation | | |
|---|---|---|---|---|
| Kinase | Motif(s) | Sites | | Protein substrate |
| cAMP-dependent | R-X-S/T | Y₇LRRASLAQLT | (SEQ ID NO: 4) | pyruvate kinase |
| Protein Kinase (PKA, | (SEQ ID NO: 2) | F₁RRLSIST | (SEQ ID NO: 5) | phosphorylase |
| cAPK) | R-R/K-X-S/T | | | kinase α-chain |
| | (SEQ ID NO: 3) | A₂₉GARRKASGPP | (SEQ ID NO: 6) | histone H1, |
| | | | | bovine |
| Casein Kinase I (CKI, | S(P)-X-X-S/T | R₄TLS(P)VSSLPGL | (SEQ ID NO: 8) | glycogen |
| CK-1) | (SEQ ID NO: 7) | D₄₃IGS(P)ES(P)TEDQ | (SEQ ID NO: 9) | synthase, rabbit |
| | | | | muscle |
| | | | | α_{S1}-casein |
| Casein Kinase II (CKII, | S/T-X-X-E | A₇₂DSESEDEED | (SEQ ID NO: 11) | PKA regulatory |
| CK-2) | (SEQ ID NO: 10) | | | subunit, R_{II} |
| | | L₃₇ESEEEGVPST | (SEQ ID NO: 12) | p34^{cdc2}, human |
| | | E₂₆DNSEDEISNL | (SEQ ID NO: 13) | acetyl-CoA |
| | | | | carboxylase |
| Glycogen Synthase | S-X-X-X-S(P) | S₆₄₁VPPSPSLS(P) | (SEQ ID NO: 15) | glycogen |
| Kinase 3 (GSK-3) | (SEQ ID NO: 14) | | | synthase, human |
| | | | | (site 3b) |
| | | S₆₄₁VPPS(P)PSLS(P) | (SEQ ID NO: 16) | glycogen |
| | | | | synthase, human |
| | | | | (site 3a) |
| Cdc2 Protein Kinase; | S/T-P-X-R/K | P₁₃AKTPVK | (SEQ ID NO: 18) | histone H1, calf |
| CDK2-cyclin A | (SEQ ID NO: 17) | | | thymus |
| | | H₁₂₂STPPKKKRK | (SEQ ID NO: 19) | large T antigen |
| Calmodulin-dependent | R-X-X-S/T | N₂YLRRRLSDSN | (SEQ ID NO: 20) | synapsin (site 1) |
| Protein Kinase II | R-X-X-S/T-V | K₁₉₁MARVFSVLR | (SEQ ID NO: 21) | calcineurin |
| (CaMK II) | | | | |
| Mitogen-activated | P-X-S/T-P | P₂₄₄LSP | (SEQ ID NO: 24) | c-Jun |
| Protein Kinase | (SEQ ID NO: 22) | P₉₂SSP | (SEQ ID NO: 25) | cyclin B |
| (Extracellular Signal- | X-X-S/T-P | V₄₂₀LSP | (SEQ ID NO: 26) | Elk-1 |
| regulated Kinase) | (SEQ ID NO: 23) | | | |
| (MAPK, Erk) | | | | |
| Abl Tyrosine Kinase | I/V/L-Y-X-X-P/F | | | |
| | (SEQ ID NO: 27) | | | |

Other illustrative protein kinase substrates are shown in Table 3.

TABLE 3
Illustrative protein kinase substrates.

| | | SEQ |
|---|---|---|
| | | ID |
| Kinase | Substrate | NO |
| cAMP-dependent protein kinase | LRRASLG (Kemptide) | 28 |
| cAMP dependent protein kinase | GRTGRRNSI | 29 |
| (PKA) | | |
| protein kinase C (PKC) | QKRPSQRSKYL | 30 |
| protein kinase Akt/PKB | RPRAATF | 31 |
| Abl kinase | EAIYAAPFAKKK | 32 |
| 5′-AMP-activated protein kinase | HMRSAMSGLHLVKRR | 33 |
| (AMPK) | | |
| C a2+/calmodulin-dependent | KKALRRQETVDAL (Autocamtide-2) | 34 |
| protein kinase | | |
| cyclin-dependent kinase 2 (cdc2) | (Ac-S)PGRRRRK | 35 |
| cyclin-dependent kinase 2 (Cdk2) | HHASPRK | 36 |
| cyclin-dependent kinase 5 (Cdk5) | PKTPKKAKKL | 37 |
| casein kinase 1 (CK1) | RRKDLHDDEEDEAMSITA | 38 |
| CK2 alpha subunit or holoenzyme | RRRDDDSDDD | 39 |
| DYRK family protein kinases | KKISGRLSPIMTEQ | 40 |
| GSK3 alpha and beta | YRRAAVPPSPSLSRHSSPHQ(pS)EDEEE | 41 |
| Src kinase | KVEKIGEGTYGVVYK | 42 |
| checkpoint kinases CHK1 and | KKKVSRSGLYRSPSMPENLNRPR | 43 |
| CHK2 | | |
| protein tyrosine kinases (PTKs) | Poly(Glu:Tyr)_{4:1} is sodium salt polymer | 44 |
| in phosphorylation assays. | with a random amino acid distribution | |
| | and a molar ratio of 4:1 for glutamic | |
| | acid:tyrosine. | |

and many kinase substrates are commercially available. Thus, for example, Table 4 lists a number of kinase substrates available from BioMol, International Lp.

TABLE 4
Illustrative commercially available kinase substrates. Available
from BiolMol International, Lp.

| Catalog # | Name | Category |
|---|---|---|
| P-216 | Abl Kinase Peptide Substrate | Abelson Murine Leukemia |
| | | Kinase (Abl) |
| P-129 | Akt Substrate | Akt/Protein Kinase B |
| P-101 | Autocamtide-2, Protein Kinase | CaMK Substrates |
| | Substrate | |
| P-148 | Biotinylated IκB Kinase Substrate Peptide | IκB Kinase (IKK) |
| P-112 | BPDEtide, cGMP-dependent Protein | PKG Substrate |
| | Kinase (PKG) Substrate | |
| P-100 | CaM Kinase IV Substrate (peptide- | CaMK Substrates |
| | γ) | |
| P-146 | Casein Kinase I Peptide Substrate | Casein Kinases |
| P-103 | Casein kinase II β (198-215), CDK | CDK and Chk Substrates and Cell |
| | (cyclin-dependent kinase) Substrate | Cycle-related Peptides |
| P-147 | Casein Kinase II Peptide Substrate | Casein Kinases |
| P-113 | Casein Kinase II Peptide Substrate | Casein Kinases |
| P-158 | Chk 1 & 2 Peptide Substrate | Checkpoint Kinase |
| | | (Chk)Checkpoint Kinases |
| SE-151 | c-Jun (1-79), JNK Substrate (rat, | Jun N-terminal Kinase (JNK) |
| | recombinant) | |
| P-195 | CREBtide Protein Kinase Substrate | PKC SubstratesPKA Substrates |
| P-149 | Crosstide | Akt/Protein Kinase B |
| P-104 | CSH103, p34^{cdc2} (CDK1)Protein | CDK and Chk Substrates and Cell |
| | Kinase Substrate | Cycle-related Peptides |
| P-121 | EGFR (661-681) T669 Peptide, | MAPK and Related Substrates |
| | MAP Kinase Substrate | |
| P-109 | EGFR Fragment (651-658), Protein | PKC Substrates |
| | Kinase Substrate | |
| P-124 | Erk1/Erk2 Peptide, MAP Kinase | MAPK and Related Substrates |
| | Kinase Substrate | |
| P-215 | Fyn Kinase Peptide Substrate | Src Family Substrates |
| P-193 | GSK Peptide Substrate II | GSK Substrates |
| P-151 | GSK-3 Peptide Substrate | GSK Substrates |
| P-106 | H1-7 (histone H1 phosphorylation | PKA Substrates |
| | site), PKA Substrate | |
| P-226 | IKK Peptide Substrate | IκB Kinase (IKK) |
| P-314 | IR0, Insulin Receptor [1142-1153] | Insulin Receptor Substrates |
| P-107 | Kemptide, Protein Kinase Substrate | PKA Substrates |
| P-217 | Lyn and Syk Kinase Peptide | Syk Family Substrates Src Family |
| | Substrate | Substrates |
| P-108 | Malantide, Protein Kinase Substrate | PKA Substrates |
| P-196 | MAPKAPK2 Peptide Substrate | MAPK and Related Substrates |
| P-117 | MARCKS psd Peptide, PKC | PKC Substrates |
| | Substrate | |
| P-110 | MBP (4-14), N-acetylated, Protein | PKC Substrates |
| | Kinase C Substrate | |
| P-114 | MLCK Substrate, Skeletal and | MLCK Substrates |
| | Smooth Muscle | |
| P-115 | MLCK Substrate, Skeletal Muscle | MLCK Substrates |
| SE-459 | Myelin Basic Protein (bovine, | Myelin Basic |
| | purified), biotinylated | Protein Miscellaneous Ser/Thr |
| | | Kinase Reagents MAPK and |
| | | Related Substrates |
| SE-458 | Myelin Basic Protein (bovine, | Myelin Basic |
| | purified), MBP | Protein Miscellaneous Ser/Thr |
| | | Kinase Reagents MAPK and |
| | | Related Substrates |
| SE-441 | Myelin Basic Protein (human, | Myelin Basic |
| | purified), MBP | Protein Miscellaneous Ser/Thr |
| | | Kinase Reagents MAPK and |
| | | Related Substrates |
| SE-310 | Myelin Basic Protein (mouse, | Myelin Basic |
| | purified), MBP | Protein Miscellaneous Ser/Thr |
| | | Kinase Reagents MAPK and |
| | | Related Substrates |
| P-194 | PAK4/AKT Peptide Substrate | p21-activated Kinase |
| | | (PAK)Akt/Protein Kinase B |
| SE-197 | PHAS-I Substrate (rat, | MAPK and Related Substrates |
| | recombinant) | |
| P-111 | PKC [Ser-25] (19-31), Substrate | PKC Substrates |
| P-155 | PKCε Pseudosubstrate Peptide, PKC | PKC Substrates |
| | Substrate | |
| P-154 | PKCδ Peptide Substrate | PKC Substrates |
| P-156 | PKCζ Peptide Substrate | PKC Substrates |
| P-304 | pp60^{c-src} C-terminal Peptide, | Src Family Substrates Peptides |
| | Tyrosine Kinase Substrate | |
| P-307 | pp60^{v-src} Autophosphorylation Site, | EGFR Substrates |
| | Tyrosine Kinase Substrate | |
| SE-308 | PRAS40 (human, recombinant) | Akt/Protein Kinase B |
| P-308 | RR-SRC, Protein Tyrosine Kinase | Src Family Substrates |
| | Substrate | |
| P-144 | S6 Ribosomal Protein Peptide | S6 Kinases |
| | Substrate | |
| P-197 | Src Peptide Substrate | Src Family Substrates |
| P-102 | Syntide-2, Protein Kinase Substrate | CaMK Substrates |
| P-123 | TH(24-33), MAP Kinase Substrate | MAPK and Related Substrates |

In certain embodiments, preferred kinase substrates include, but are not limited to substrates for histidine kinases, serine kinases, threonine kinases, and tyrosine kinases and/or the corresponding phosphatases. Many substrates for these kinases are well known to those of skill in the art. In addition, methods are well known for identifying such substrates. Thus, for example, the program PREDIKIN can be used to predict substrates for serine/threonine protein kinases based on the primary sequence of a protein kinase catalytic domain. Rules for substrate prediction are based on sequences similar to those that would be found by an oriented peptide library experiment, in known natural substrates and by modeling using the Insight II software package (Accelrys). PREDIKIN is described in detail by Ross et al. (2003) Proc. Natl. Acad. Sci., USA, 100(1): 74-79, which is incorporated herein by references. Similar programs for the identification of other kinase substrates are known to those of skill in the art.

In addition, many screening systems are known and available for identifying kinase substrates. In one approach, for example, anti-phosphotyrosine antibodies are used to screen tyrosine-phosphorylated cDNA expression libraries (see, e.g., Lock et al. (1998) EMBO J. 17(15): 4346-4357, which is incorporated herein by reference). Another approach utilized in vivo labeling of proteins with “light” (¹²C-labeled) or “heavy” (¹³C-labeled) tyrosine. This stable isotope labeling in cell culture method enables the unequivocal identification of tyrosine kinase substrates, because peptides derived from true substrates give rise to a unique signature in a mass spectrometry experiment (see, e.g., Ibarrola et al. (2004) J. Biol. Chem., 279(16): 15805-15813, which is incorporated herein by reference). These approaches are readily automated and amenable to high throughput screening systems (HTS).

Moreover, as indicated above, a number of substrates are already known and no screening is required for their identification. Thus, for example, a number of tyrosine kinase substrates and the associated phosphorylation site are shown in Table 5.

TABLE 5
Illustrative Tyrosine Kinase substrates

| | Phosphorylation | | Phosphorylation |
|---|---|---|---|
| Substrate | Site | Substrate | Site |
| KDR | Tyr996 | PLCg | Tyr771/775 |
| STAT3 | Tyr705 | T-cell activation antigen | Tyr217 |
| cdc2 | Tyr15 | T-cell Receptor Zeta chain | Tyr152 |
| JAK1 | Tyr1022/1023 | ERK5 | Tyr215/220 |
| KDR | Tyr1054/1059 | GSK3 | Tyr284 |
| Paxillin | Tyr31 | JNK1 | Tyr190 |
| Pyk2 | Tyr402 | TrkC | Tyr705 |
| Shc | Tyr317 | Zinc Finger Protein 145 | Tyr70 |
| STAT1 | Tyr701 | TIF | Tyr495 |
| TrkA | Tyr490 | c-Kit (Y900 | 64 |
| TrkA | Tyr785 | PTP1B | Tyr66 |
| Tyk2 | Tyr1054/1055 | SHP-2 (Try542 | 63 |
| Zap70 | Tyr493 | PI3K | Tyr688 |
| STAT6 | Tyr641 | Src | Tyr416 |
| HER2 | Tyr1248 | c-FGR | Tyr412 |
| STAT5 | Tyr694 | EGFR | Tyr1173 |
| CTD | Tyr | ER a | Tyr537 |
| FAK | Tyr577 | IRS1 | Tyr891 |
| STAT4 | Tyr693 | IRS2 | Tyr766 |
| PDGFR | Tyr775 | JAK2 | Tyr1008 |
| STAT2 | Tyr690 | PTEN | Tyr315 |
| JAK1 | Tyr1023 | c-Cbl | Tyr700 |
| Liver Glycogen | Tyr637 | Dynamin I/II | Tyr231 |
| Synthase | | | |
| NLK-1 | Tyr181 | P62Dok | Tyr398 |
| PDGFR | Tyr771 | R-Ras | Tyr66 |
| Signal Transduction | Tyr160 | PTEN | Tyr336 |
| Protein | | | |
| TLE2 | Tyr226 | VEGFR1 | Tyr1213 |
| beta-adrenergic | Tyr350 | VEGFR2 | Tyr1212 |
| receptor | | | |
| CSBP1 | Tyr182 | Zap70 | Tyr319 |
| doublecortin | Tyr345 | c-Cbl | Tyr774 |
| HER2 | Tyr1248 | Met | Tyr1349 |
| Insulin Receptor | Tyr992 | Met | Tyr1356 |
| Precursor | | | |
| HEK8 | Tyr596 | VEGFR2 | Tyr801 |
| Met | Tyr1253 | FcgammaRIIB | Tyr292 |
| MBP | Tyr117/124 | Ret | Tyr905 |

In various embodiments, the substrates for protein/peptide kinases and/or phosphatases typically range in length from about 4 amino acids up to about 200, 100 or 50 amino acids, more preferably from about 4 amino acids or six amino acids up to about 30, 40, or 50 amino acids, most preferably from about 4, 6, or 8, amino acids up to about 16, 20, 25, 30, 35, or 40 amino acids. In certain embodiments, the kinase substrate comprises one phosphorylation site. In certain embodiments, the kinase substrate comprises more than one phosphorylation site (e.g., at least 2, 3, 4, 5, 6, 8, 10, 12, or 20 phosphorylation sites). In certain embodiments, the substrate will comprise 1, 2, 3, 4, 5, 8, or 10, amino acids found on each side of the phosphorylation site in the native substrate.

As indicated, for essentially any kinase, there also exists a corresponding phosphatase (e.g., to dephosphorylate the substrate at the same, or different, site). In certain embodiments a kinase can act as a kinase at one site on a substrate and a phosphatase at a different site on that substrate and/or on a different substrate. Thus, in various embodiments, kinase substrates can also act as a phosphatase substrates.

In addition, the substrates that can be modified by enzyme or other chemical processes to for example, add or remove extra functional chemical groups or chemical structures to the existing substrate. Thus, in certain embodiments various chemical modifications (e.g., acetylation, blocking, amidation, formylation, sulfonation, methylation, etc.) are performed on one or more of the residues comprising the substrate, In certain embodiments the substrates can comprise one or more non-naturally occurring amino acid residues (e.g., 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like).

The foregoing kinase and/or phosphatase substrates are intended to be illustrative and not limiting. Using the teachings provided herein, other kinase substrates will be readily available to one of skill in the art for use in the methods, compositions and devices described herein.

### II. Fabrication of Surface for SERS Kinase/Phosphatase Assay Arrays.

In various embodiments, the kinase and/or phosphatase substrate(s) are attached to a nanoparticle or nanoparticles and/or to a nanoscale feature comprising a surface, preferably a Raman-active surface. In one illustrative approach, a surface comprising a nanopyramids (a nanopyramid array) is provided. First, 100-500 nm thick polysilicon layer is grown on the surface of a single crystal silicon wafer. The surface of the wafer is then treated with the plasma of the mixture of HBr and O₂ for less than 10 seconds. In this step a nanoscale oxide island array forms on the polysilicon surface. Third, wafer surface is then etched, e.g., by HBr plasma for 10˜20 second to form nanopillar arrays. The oxide island layer is then removed by, e.g., SF₆ plasma etching. Then the polysilicon surface with nanopillar patterns is etched by, e.g., HBr plasma for about 1˜2 minutes. The polysilicon nanopyramid patterns naturally form on the wafer surface. After surface metallization, the nanopyramid array can be used as the SERS substrate. The process flow is illustrated in FIG. 7. Where nanopillars rather than nanopyramids are desired the final etch can be reduced or eliminated.

In certain embodiments, surfaces comprising nanoscale features can be batch fabricated using the methods described by Liu and Lee (2005) Appl. Phys. Letts., 87: 074101, which is incorporated herein by reference.

In this approach, nanostructures are fabricated, e.g., on a silicon or glass substrate using conventional lithography and etching methods. The best master copy is chosen, and repeatable PDMS-based soft lithography is applied in conjunction with a simple metal deposition on the replicated nanostructures, which allows economical mass production of identical SERS active sites on polymer substrates. The background noise of Raman signals from the polymer substrate is avoided since the deposition of metal thin film (e.g., Ag or Au) for the formation of nanowell SERS structures blocks excitation light sources to pick up extra Raman signals from PDMS polymer substrate.

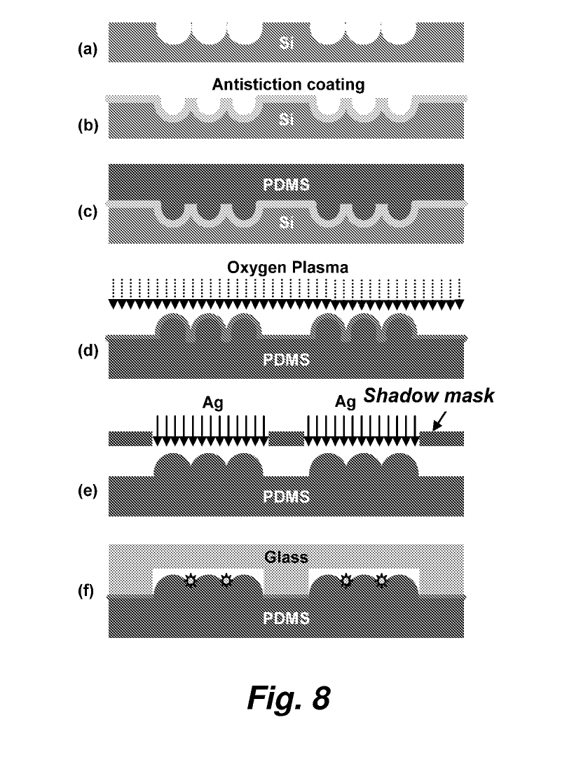

This process is schematically illustrated in FIG. 8. As shown therein, nanowells are fabricated on silicon as a master copy for a PDMS SERs substrate (see, panel (a)). An antistiction coating is applied to the master copy of nanowells (panel b). Soft lithography of the nanowells is performed using a PDMS polymer (panel c). The surface is treated with oxygen plasma (panel d), followed by selective deposition of a thin film metal (e.g., Ag) layer for SERS active sites using a shadow mask (panel e), and the resulting SERs structure is integrated into a glass microfluidic channel (panel f).

As illustrated in various embodiments, a simple shadow masking process for selective thin film metal deposition on nanostructured PDMS substrate provides an effective integration solution to bond with a glass-based microfluidic channel array chip (see, e.g., FIGS. 8, 9, and 10).

In various other embodiments, known methods of assembling nanoparticles on surface such as silicon (see, e.g., Liu and Green (2004) J. Mater. Chem. 14: 1526) or polymers (see, e.g., Lu et al. (2005) Nano Lett. 5: 5) as well as E-beam fabricated nanoparticle arrays (see, e.g., Liao et al. (1981) Chem. Phys. Lett. 82: 355) can be utilized. In certain embodiments, the nanoparticles can be preformed and electrostatically, thermally, ionically or chemically affixed to an underlying surface. In various embodiments the nanoparticles can include nanopillars, nanorods, nanopyramids, nanowires, nanospheres, a nanocrescents, nanohorns, nanotubes, nanotetrepods, a single- or multi-layered nanodisks, and the like.

In various embodiments the nanofeatures range in size from about 10 nm to about 200 nm, more preferably from about 20 nm to about 100 nm, still more preferably from about 30 nm to about 50 or 80 nm. In various embodiments the average spacing between nanofeatures ranges from about 2 nm to about 100 nm, still more preferably from about 4 nm to about 50 or 80 nm. In one illustrative embodiment, the nanoscale features have an average dimension (e.g., diameter) of about 35-45 nm and an average spacing of about 40 to about 50 nm. In certain embodiments the nanoscale features have a center to center distance that ranges from about 10, 15, 20, or 25 nm to about 100, 150, 200, 250, 300, 350, 400, 450, or 500 nm. In certain embodiments the center to center distance of the features ranges from about 50 or 75 nm to about 100 nm, 150 nm, or 200 nm.

In various embodiments when incorporated into a microfluidic chamber, chamber volumes can be less than about 10 μl, or 1 μl, or 100 nL, preferably less than about 10 nL or 1 nL, still more preferably less than about 0.1 nL, or 0.01 nL.

The foregoing methods and embodiments are intended to be illustrative and not limiting. Using the teachings provided herein other surfaces comprising nanoscale features can be fabricated by one of skill in the art.

While the Raman active surface comprising nanoscale features (e.g., nanopyramids) is illustrated herein as a surface comprising gold, it will be recognized that the surface can be fabricated of other materials. Such materials, include for example, noble metal, a noble metal alloy, a noble metal composite, a nobel metal nitrate, a noble metal oxide, and the like.

In certain embodiments the Raman-active surface is comprised of a metal or a semiconductor material. Suitable materials include, but are not limited to metals (e.g., gold, silver, copper, tungsten, platinum, titanium, iron, manganese, and the like, or oxides, nitrides, or alloys thereof), semiconductor materials (e.g., CdSe, CdS, and CdS or CdSe coated with ZnS, and the like), multi-layers of metals and/or metal alloys, and/or metal oxides or nitrides, polymers, carbon nanomaterials, magnetic (e.g., ferromagnetite) materials, and the like. In certain embodiments materials comprises one or more of the following: tungsten, tantalum, niobium, Ga, Au, Ag, Cu, Al, Ta, Ti, Ru, Ir, Pt, Pd, Os, Mn, Hf, Zr, V, Nb, La, Y, Gd, Sr, Ba, Cs, Cr, Co, Ni, Zn, Ga, In, Cd, Rh, Re, W, Mo, and oxides, nitrides, alloys, and/or mixtures and/or sinters thereof. Other materials useful in the practice of the invention include, but are not limited to ZnS, ZnO, Ti02, AgI, AgBr, HgI2, PbS, PbSe, ZnTe, CdTe, In₂S₃, In₂Se₃, Cd₃P₂, Cd₃As₂, InAs, GaAs, and the like.

### III. Attachment to and Patterning of Kinase/Phosphatase Substrate(s) to Nanoparticles, or Raman Active Substrate.

The kinase and/or phosphatase substrates can be attached to nanoparticle(s) or to features present on a surface (e.g. a Raman active surface) by any of a number of methods well known to those of skill in the art. For example, in certain instances, the kinase and/or phosphatase substrate(s) can simply be adsorbed to the surface.

However, to maximize access of the kinase and/or phosphatase substrate(s) to the kinase(s)/phosphatase(s) in an assay, it is often desirable to covalently attach the kinase and/or phosphatase substrate to the nanoparticle of nanoscale features on a surface directly (e.g., through a functional group) or through a linker.

For example, in certain embodiments the kinase and/or phosphatase substrates are tethered onto the a gold nanoscale feature using, a cysteine group at the terminus of the substrate (e.g., peptide) to attach the substrate to the gold surface, relying on the gold-thiol reaction to form a covalent bond. In various embodiments the array surface and/or the kinase and/or phosphatase substrate can derivatized with, for example, amine, carboxyl groups, alkyl groups, alkyene groups, hydroxyl groups, or other functional groups so the peptide (or other substrate) can be linked directly to the surface or coupled through a linker. In other embodiments, the surface can be functionalized, e.g. with amine, carboxyl, or other functional groups for attachment to the kinase and/or phosphatase substrate(s).

Suitable linkers include, but are not limited to hetero- or homo-bifunctional molecules that contain two or more reactive sites that may each form a covalent bond with the respective binding partner (kinase/phosphatase substrate, surface, or functional group thereon, etc.). Linkers suitable for joining such moieties are well known to those of skill in the art. For example, a protein molecule can readily be linked by any of a variety of linkers including, but not limited to a peptide linker, a straight or branched chain carbon chain linker, or by a heterocyclic carbon linker. Heterobifunctional cross-linking reagents such as active esters of N-ethylmaleimide have been widely used to link proteins to other moieties (see, e.g., Lerner et al. (1981) Proc. Nat. Acad. Sci. (USA), 78: 3403-3407; Kitagawa et al. (1976) J. Biochem., 79: 233-236; Birch and Lennox (1995) Chapter 4 in Monoclonal Antibodies: Principles and Applications, Wiley-Liss, N.Y., and the like).

In certain embodiment, the kinase and/or phosphatase substrate can be joined to the surface utilizing a biotin/avidin interaction. In certain embodiments biotin or avidin, e.g. with a photolabile protecting group can be affixed to the surface and/or kinase/phosphatase substrate(s). Irradiation of the surface in the presence of the desired kinase and/or phosphatase substrate bearing the corresponding avidin or streptavidin, or biotin, results in coupling of the substrate to the surface.

Where the surface and/or the kinase and/or phosphatase substrate bear reactive groups or are derivatized to bear reactive groups numerous coupling methods are readily available. Thus, for example, a free amino group is amenable to acylation reactions with a wide variety of carboxyl activated linker extensions that are well known to those skilled in the art. Linker extension can performed at this stage to generate terminal activated groups such as active esters, isocyanates, maleimides, and the like. For example, reaction of the peptide or amino-derivatized surface with one end of homobifunctional N-hydroxysuccinimide esters of bis-carboxylic acids such as terephthalic acid will generate stable N-hydroxysuccinimide ester terminated linker adducts that useful for conjugation to amines. Linker extension can also be accomplished with heterobifunctional reagents such as maleimido alkanoic acid N-hydroxysuccinimide esters to generate terminal maleimido groups for subsequent conjugation to thiol groups. An amino-terminated linker can be extended with a heterobifunctional thiolating reagent that reacts to form an amide bond at one end and a free or protected thiol at the other end. Some examples of thiolating reagents of this type which are well known in the art are 2-iminothiolane (2-IT), succinimidyl acetylthiopropionate (SATP) and succinimido 2-pyridyldithiopropionate (SPDP). The incipient thiol group is then available, after deprotection, to form thiol ethers with maleimido or bromoacetylated moieties or to interact directly with a gold surface. In various embodiments the amino group, e.g., of an amino-terminated linker can be converted a diazonium group and hence the substance into a diazonium salt, for example, by reaction with an alkali metal nitrite in the presence of acid, which is then reactive with a suitable nucleophilic moiety, such as, but not limited to, the tyrosine residues of peptides, and the like. Examples of suitable amino-terminated linkers for conversion to such diazonium salts include, but are not limited to aromatic amines (anilines), and may also include the aminocaproates and similar substances referred to above. Such anilines can readily be obtained by substituting into the coupling reaction between the an available hydroxyl group and an N-protected amino acid, as discussed above, the corresponding amino acid wherein the amino group is comprised of an aromatic amine, that is, an aniline, with the amine suitably protected, for example, as an N-acetyl or N-trifluoroacetyl group, which is then deprotected using methods well-known in the art. Other suitable amine precursors to diazonium salts will be suggested to one skilled in the art of organic synthesis.

Another favored type of heterobifunctional linker is a mixed active ester/acid chloride such as succinimido-oxycarbonyl-butyryl chloride. The more reactive acid chloride end of the linker preferentially acylates amino or hydroxyl groups, e.g., on the peptide to give N-hydroxysuccinimidyl ester linker adducts directly.

Yet another type of terminal activated group useful in the present invention is an aldehyde group. Aldehyde groups may be generated by coupling a free hydroxyl (e.g. on a peptide or derivatized nanocrescent) with an alkyl or aryl acid substituted at the omega position (the distal end) with a masked aldehyde group such as an acetal group, such as 1,3-dioxolan-2-yl or 1,3-dioxan-2-yl moieties, followed by unmasking of the group using methods well-known in the art. In various embodiments alkyl or aryl carboxylic acids substituted at the omega position with a protected hydroxy, such as, for example, an acetoxy moiety, may be used in coupling reactions, followed by deprotection of the hydroxy and mild oxidation with a reagent such as pyridinium dichromate in a suitable solvent, preferably methylene chloride, to give the corresponding aldehyde. Other methods of generating aldehyde-terminated substances will be apparent to those skilled in the art.

In various embodiments, multiple kinase and/or phosphatase substrates are attached to the Raman-active surface (e.g., surface comprising nanoscale features). In various embodiments at least five, preferably at least 10, more preferably at least 20, 50, or 100, and most preferably at least 100, 500, 1,000, 10,000, 50,000, or 100,000 different kinase and/or phosphatase substrates are attached to a surface.

In certain embodiments, the surface provides a high density array of kinase and/or phosphatase substrates. In various embodiments such an array can comprise at least 100 or at least 200 different substrates/cm², preferably at least 300, 400, 500, or 1000 different substrates/cm², and more preferably at least 1,500, 2,000, 4,000, 10,000, or 50,000, or 100,000 different substrates/cm².

Methods of patterning molecules on surfaces at high density are well known to those of skill in the art. Such methods include, for example, the use of high density microarray printers which are essentially spotting printers (see, e.g., Heller (2002) Annu Rev Biomed Eng. 4: 129-153). Other microarray printers utilize “on-demand” piezoelectric droplet generators (e.g., inkjet printers) (see, e.g., U.S. Pat. Nos. 6,395,562; 6,365,378; 6,228,659 and 5,338,688 and WO publications WO 95/251116 and WO/2003/028868 which are incorporated herein by reference. Other approaches involve de novo synthesis in place (see, e.g., Fodor et al. (1991) Science, 251: 767-773, and U.S. Pat. Nos. 6,269,846, 6,271,957, and 6,480,324 which are incorporated herein by reference). A number of array printers are commercially available (see, e.g., VERSA Mini Spot-printing Workstation from Aurora Biomed, BioOdyssey™ Calligrapher™ MiniArrayer from Bio-Rad, QArray mini from Genetix, BioRobotics MicroGrid from Genomic Solutions, OmniGrid Accent from Genomic Solutions, and the like).

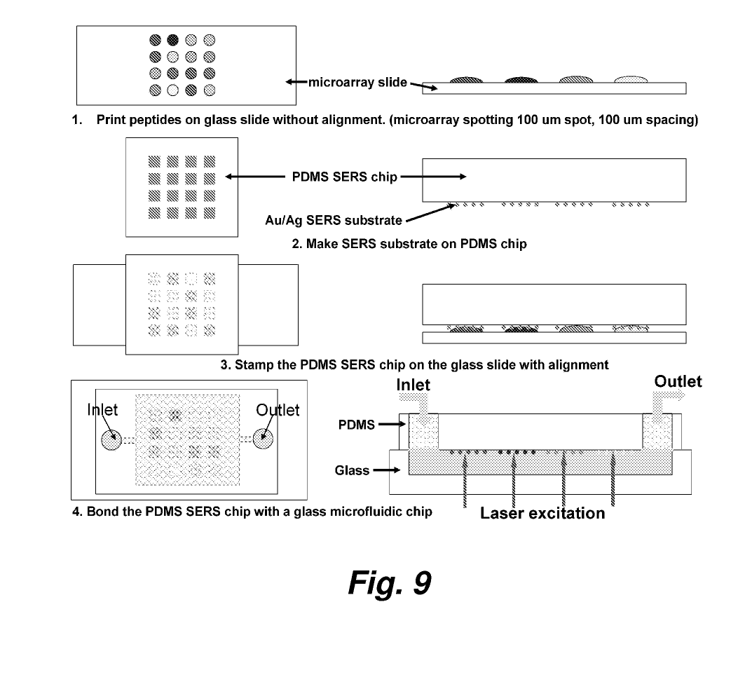

The kinase and/or phosphatase substrates can be patterned directly on the Raman active surface (e.g., the nanopillar or nanopyramid array) using the methods described above. Alternatively, the kinase and/or phosphatase substrates can be patterned on a different surface, e.g., a glass slide and then transferred to the Raman active surface by contacting the Raman active surface to the printed array thereby transferring the kinase and/or phosphatase substrate molecules from the previously printed surface to the Raman active surface (see, e.g., FIG. 9).

### IV. Assay Formats and Sample Delivery

In various embodiments a number of different kinase assay formats are contemplated. For example, where it is desirable, to detect and/or quantify a single species of kinase and/or phosphatase in a sample, Raman active surface can be provided that comprises a single species of kinase and/or phosphatase substrate. In certain embodiments the surface can be partitioned for application of different samples at different locations. In other embodiments, it is desirable to detect and/or quantify different kinases and/or phosphatases and a surface can be provided that comprises a plurality of kinase and/or phosphatase substrates. In certain embodiments the multi-species surface can be partitioned for simultaneous detection of different samples.

In certain embodiments, any of the surfaces comprising one or more than one species of kinase and/or phosphatase substrate, can optionally include one or more positive and/or negative controls. In certain embodiments, a negative control comprises one or more molecules of the same species as the kinase substrates and/or phosphatase, but lacking a phosphorylation site for a particular kinase/phosphatase and/or for any kinase/phosphatase expected to be present in the assay. In certain embodiments, a positive control comprises one or more molecules of the same species as the kinase and/or phosphatase substrates, but containing a phosphorylation site for a kinase and/or phosphatase known to be present in the assay. In certain embodiments, the positive or negative controls may comprise molecules of a species different than the kinase and/or phosphatase substrate(s) on the surface.

The kinase and/or phosphatase assays described herein can be performed on any of a number of different samples. For example, in screening systems for the identification of kinase inhibitors, cells/cell lines and/or lysates thereof or appropriate buffer systems comprising the kinase(s) of interest can be contacted/administered one or more test compounds. The samples derived therefrom can then be screened for kinase activity thereby identifying which test compounds are effective, e.g., as kinase inhibitors and/or phosphatase agonists, and which kinase/phosphatase they inhibit/agonize.

In various diagnostic embodiments the kinase and/or phosphatase presence, and/or concentration, and/or activity is determined in a biological sample. The biological sample can include essentially any biomaterial that it is desired to assay. Such biomaterials include, but are not limited to biofluids such as blood or blood fractions, lymph, cerebrospinal fluid, seminal fluid, urine, oral fluid and the like, tissue samples, cell samples, tissue or organ biopsies or aspirates, histological specimens, and the like.

In certain embodiments the raw cell lysate can be directly applied on the SERS microarray and the measurement can be done during the incubation. To ensure consistent solution height of aqueous samples in measurement, microfluidic reaction chamber can be bonded with the SERS microarray. Samples are introduced into the reaction chamber through microfluidic channels. The total sample volume can be reduced to sub-microliter volumes.

In various embodiments the biological sample (e.g., cell lysate or derivative thereof) is applied to the Raman active surface comprising the kinase and/or phosphatase substrate(s). This can be accomplished in certain embodiments by flowing the sample through a microfluidic chamber containing/comprising the Raman active surface. In certain embodiments the microchamber is mounted on a thermal plate (e.g., at 37° C.) on an inverted Raman microscope with darkfield illumination for nanoparticle visualization. The excitation laser is focused on the various regions of the Raman active surface, e.g., by a microscopy objective lens. The SERS signal is collected by the same objective lens and analyzed by a spectrometer.

### V. Detection and Automated Detection System(s)

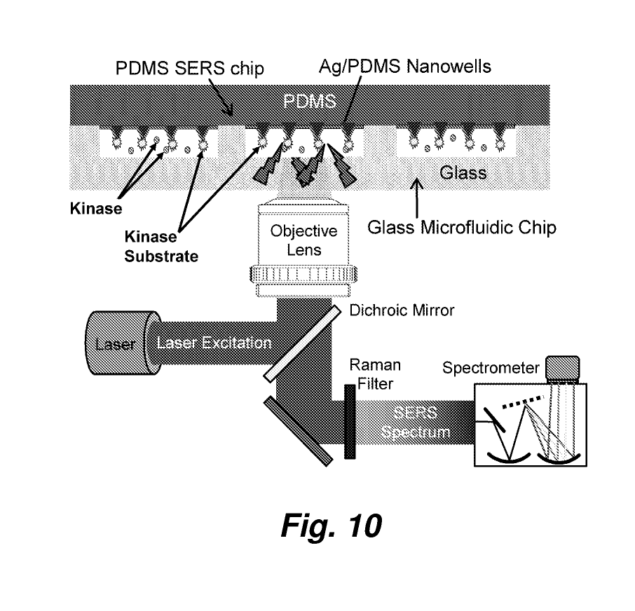

A variety of detection units of potential use in Raman spectroscopy are known in the art and any known Raman detection unit may be used. A non-limiting example of a Raman detection unit is disclosed in U.S. Pat. No. 6,002,471. In this example, the excitation beam is generated by either a Nd:YAG laser at 532 nm (nanometer) wavelength or a Ti:sapphire laser at 365 nm wavelength. Pulsed laser beams or continuous laser beams may be used. The excitation beam passes through confocal optics and a microscope objective, and may be focused onto a substrate containing attached biomolecule targets. Raman emission light target(s) can be collected by the microscope objective and the confocal optics, coupled to a monochromator for spectral dissociation. The confocal optics can include a combination of dichroic filters, barrier filters, confocal pinholes, lenses, and mirrors for reducing the background signal. Standard full field optics can be used as well as confocal optics (see, e.g., FIG. 10).

The Raman emission signal can be detected by a Raman detector. The detector can include an avalanche photodiode interfaced with a computer for counting and digitization of the signal. Where arrays of target(s) are to be analyzed, the optical detection system may be designed to detect and localize Raman signals to specific locations on a chip or grid. For example, emitted light may be channeled to a CCD (charge coupled device) camera or other detector that is capable of simultaneously measuring light emission from multiple pixels or groups of pixels within a detection field.

Other examples of Raman detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source is a 514.5 nm line argon-ion laser from SpectraPhysics, Model 166, and a 647.1 nm line of a krypton-ion laser (Innova 70, Coherent).

Various excitation sources include, but are not limited to, a nitrogen laser (Laser Science Inc.) at 337 nm and a helium-cadmium laser (Liconox) at 325 nm (U.S. Pat. No. 6,174,677). The excitation beam can be spectrally purified with a bandpass filter (Corion) and may be focused on a substrate 140 using a 6.times. objective lens (Newport, Model L6X). The objective lens can be used to both excite the indicator(s) and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) can be used to reduce Rayleigh scattered radiation. Alternative Raman detectors include, but are not limited to, an ISA HR-320 spectrograph equipped with a red-enhanced intensified charge-coupled device (RE-ICCD) detection system (Princeton Instruments). Other types of detectors may be used, such as charged injection devices, photodiode arrays or phototransistor arrays.

In certain embodiments the scattering image and spectrum of the kinase and/or phosphatase substrates are acquired using a dark-field microscopy system with a true-color imaging camera and a spectrometer. In one illustrative embodiment, the microscopy system consists of a Carl Zeiss Axiovert 200 inverted microscope (Carl Zeiss, Germany) equipped with a darkfield condenser (1.2<NA<1.4), a true-color digital camera (CoolSNAP cf, Roper Scientific, NJ), and a 300 mm focal-length and 300 groove/mm monochromator (Acton Research, MA) with a 1024×256-pixel cooled spectrograph CCD camera (Roper Scientific, NJ).

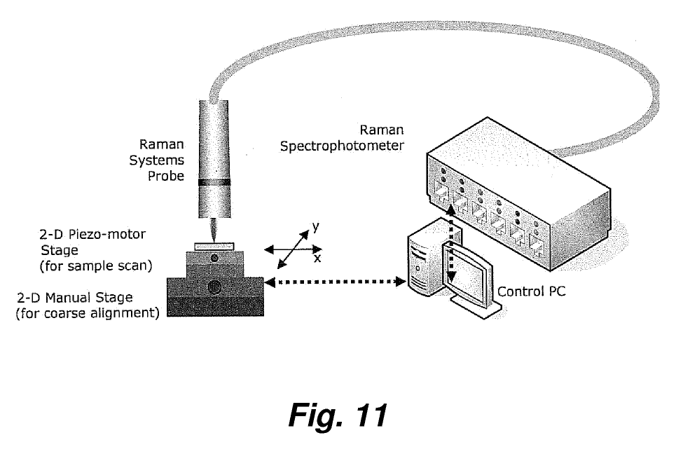

One detection system is schematically illustrated in FIG. 11. As shown therein, the detection system comprises an x-y scanning sample stage, Raman detection probe, spectrophotometer and control computer. The Raman detection probe comprises a laser light delivery fiber, an objective lens, a long-pass optical filter and a Raman scattering light collection fiber. The SERS microarray chip is mounted on the scanning stage and the SERS signal of the peptides at each spot is measured by the fixed Raman detection probe while the stage scan and data acquisition are synchronized by the control computer.

### VI. Kits.

In another embodiment this invention provides kits for practice of the methods described herein. The kits typically comprise SERs array comprising a plurality of kinase and/or phosphatase substrates as described. In certain embodiments the SERs array can be provided encased in a microfluidic chamber, e.g., as a component of a microfluidic cassette for use in a SERs assay device.

In various embodiments the kits, optionally include devices (e.g., syringe, swab, etc.) and or reagents (e.g., diluents and/or buffers) for the collection and/or processing of a biological sample.

In addition, the kits optionally include labeling and/or instructional materials providing directions (i.e., protocols) for the practice of the methods described herein. In certain embodiments the instructional materials describe the use of one or more devices described herein to detect and/or quantify the presence or activity of a kinase and/or phosphatase.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Fabrication and Use of SERs Microarray

Fabrication of Nano Pyramid SERS Substrate

Starting with a single crystal silicon wafer, a 300 nm thick thin layer of poly-crystal silicon was deposited on the polished top surface of the silicon wafer. Microscale devices can be patterned on the poly-silicon surface using photolithography. After patterning the silicon wafer was etched in a plasma assisted reactive ion etcher. The etching process to make the nano pyramid SERS substrate was different from those used in conventional silicon film etching. At first, the native oxide layer on the poly silicon film was stripped off by using SF₆ plasma etching for 10 seconds. Next, a mixture of O₂ and HBr gases was flowed in the RF plasma etching chamber for 7 seconds to define nanoscale oxide islands on the top of poly silicon film surface. These nanoscale oxide islands were created by the simultaneous etching and oxidation process. The average diameter of the oxide islands was about 20 nm and the spacing distance between adjacent oxide islands was dependent on the mixing ratio of O₂ and HBr. Then the poly silicon film was be etched by pure HBr plasma for 10˜20 seconds to form short nanopillar arrays. As the nanoscale oxide islands serve as the etching mask, the nanopillar etching had excellent directionality. Subsequently, the oxide island layer was removed by SF₆ plasma etching and the silicon nanopillars were exposed. Last, the polysilicon surface with nanopillar patterns was isotropically etched by HBr plasma for 1˜2 minutes. The polysilicon nanopyramid patterns formed on the wafer surface. After surface metallization with 50-80 nm gold or silver thin film, the nanopyramid array was ready for use as a SERS substrate. The process flow is illustrated in FIG. 7.

Detections of Purified Cellular Src Kinase

Src kinase SERS probes were tested and calibrated using purified p60 cellular Src kinase first. The real time peptide SERS spectra were recorded in the reaction with 10 nM Src kinase at 37° C. The intensity of phenyl ring breathing peak 1004 cm⁻¹ increased significantly within 10 minutes of phosphorylation reaction. The phosphorylation level was defined as the normalized ratio of peak intensities between 1004 cm⁻¹ and 1260 cm⁻¹. The 1260 cm⁻¹ peak was associated with the cysteine residue and its intensity had negligible variance throughout the reactions. The initial phosphorylation level before reactions was defined as unity and increased more than 6-fold after the reactions with 10 nM Src kinase. The real time phosphorylation level in the reactions was characterized with various concentrations of Src kinase. The phosphorylation rate was dependent on the kinase concentration. The phosphorylation level saturated at around 7 for high concentration of Src kinase and the minimal detectable concentration is above 10 pM. The reaction constants of the Src kinase at different concentration can be calculated using the Michaelis-Maten model.

Detections of Kinase Inhibitor

Kinase inhibition is the most effective and direct way to interfere with cellular signaling pathways, and many cancer therapeutics are based on kinase inhibitors. Different Src kinase inhibitors were tested using the peptide-conjugated nanoprobe assay. The phosphorylation level of the Src kinase with the addition of the inhibitors PP2, PP3 and SU5656 was tested. The phosphorylation level decreases significantly with the increasing concentration of inhibitors. The IC50 concentrations for the three inhibitors were characterized respectively.

Detections of Kinase in Crude Cell Lysate

Various 3T9 mouse fibroblast cells were lysed and the cell lysate was directly mixed with the peptide-nanoparticle conjugates after removal of membrane debris. The SERS spectra on single nanoparticles was monitored before and after the introduction of the cell lysate. The Src phosphorylation in the wild type 3T9 cell lysate showed a mild level, while in the Src transfected 3T9 cell lysate, the phosphorylation level became 3 times higher. Similar inhibitor testing was also carried out. The wild type and Src-transfected 3T9 cells are cultured with the addition of the inhibitors in various concentrations.

In order to further confirm that the peptide-conjugated nanoprobes do not generate false results in irrelevant samples, Src-deficient cell line, SYC cell lysates were used. The phosphorylation measurements were carried out in wild type, Src-knock out and inhibitor-treated SYC cell lysates. The Src-free sample will not generate considerable phosphorylation level due to the high specificity of the nanoprobes even though many other active kinase may be present in the lysate.

SERS Spectroscopy

A microscopy system with Raman spectrometer was used to acquire Raman scattering spectra from single nanocrescents. The system consisted of a Carl Zeiss Axiovert 200 inverted microscope (Carl Zeiss, Germany) equipped with a digital camera and a 300 mm focal-length monochromator (Acton Research, MA) with a 1024×256-pixel cooled spectrograph CCD camera (Roper Scientific, NJ). A 785 nm semiconductor laser was used in our experiments as the excitation source of Raman scattering, and the laser beam was focused by a 40× objective lens on the nanocrescent. The excitation power was measured by a photometer (Newport, Calif.) to be ˜0.8 mW. The Raman scattering light was then collected through the same optical pathway through a long-pass filter and analyzed by the spectrometer.

Peptide Synthesis.

401 mg (0.277 mmol) of Rink Amide AM polystyrene resin (loading 0.69 mmol/g) was added to a 12 mL fitted syringe and swollen with N-methylpyrrolidinone (NMP) (4 mL). The Fmoc protecting group was removed by treatment with 1:2:2 piperidine/NMP/CH₂Cl₂ solution (3 mL) for 30 min, and the resin was filtered and washed with NMP (3×3 mL) and CH₂Cl₂ (3×3 mL). To load the amino acid residues, the resin was subjected to repeated cycles of coupling conditions, followed by washing (5×3 mL NMP, 5×3 mL CH₂Cl₂), Fmoc deprotection [treatment with 1:2:2 piperidine/NMP/CH₂Cl₂ solution (3 mL) for 30 min], and washing again with NMP (5×3 mL) and CH₂Cl₂ (5×3 mL). The first amino acid residue was loaded by addition of a preformed solution of Fmoc-Cys(Trt)-OH (1.17 g, 2.00 mmol), PyBOP (1.04 g, 2.00 mmol), and HOBt (270 mg, 2.00 mmol) in 1:1 NMP/CH₂Cl₂ (2 mL) onto the resin and the resulting slurry was stirred for 5 min on a wrist-action shaker, followed by addition of i-Pr₂EtN (0.55 mL, 4.0 mmol). The reaction was allowed to proceed for 5 h. The resin was then filtered, washed (5×3 mL NMP, 5×3 mL CH₂Cl₂), and dried under high vacuum. The loading of Cys was determined to be 0.60 mmol/g (78% yield). Successive couplings were achieved either by method A or method B. Method A consisted of addition of a preformed solution of Fmoc-protected amino acid in NMP/CH₂Cl₂ (1:1, 2 mL), followed by addition of i-Pr₂EtN (0.55 mL, 4.0 mmol). The reactions were allowed to proceed for at least 4 h. Method B consisted of subjection of the resin to a 0.4 M solution of the suitably protected acid, which had been pre-activated by incubation with DIC (130 μL, 0.84 mmol) and HOBt (108 mg, 0.800 mmol) in DMF (2 mL) for 10 min. The coupling was allowed to proceed for 4 h. After each coupling the resin was filtered and washed (NMP: 5×3 mL, CH₂Cl₂: 5×3 mL), followed by removal of the Fmoc protecting group. After coupling and deprotection of the final amino acid residue, the aminovaleric acid linker was added by subjection of the resin to a 0.4 M solution of Fmoc-S-Ava-OH (272 mg, 0.800 mmol) which had been pre-activated by incubation with DIC (120 μL, 0.80 mmol) and HOBt (108 mg, 0.800 mmol) in NMP (1 mL) for 10 min. The coupling was allowed to proceed overnight. The resin was filtered and washed (5×3 mL NMP, 5×3 mL CH₂Cl₂), the Fmoc protecting group was removed, and the resin washed again. The reaction was allowed to proceed for 6 h, the coupling procedure was repeated once more and the reaction was allowed to proceed overnight. The substrate was cleaved from the resin by incubation with a solution of 94:2:2:2 TFA/triisopropylsilane/H₂O/ethanedithiol (3 mL) for 2 h, purified using preparatory C18 reverse-phase HPLC (CH₃CN/H₂O-0.1% TFA, 5-95% for 50 min, 20 mL/min, 220/254/280 nm detection for 100 min, t_{R}=24.3 min), and lyophilized. MS (MALDI), m/z calcd for C₇₈H₁₁₆N₁₉O₁₇S: 1622.85. Found: m/z 1623.90.

The detection scheme for the de-phosphorylation process by phosphatase enzymes is similar to that in kinase detections while in a reversed way. The phosphatase substrate peptides have phosphate groups which will be deprived by active phosphatase enzymes. In this case, the dephosphorylation site on the substrate peptide will lose the negative charge and move away from the SERS substrate surface and the Raman scattering enhancement will become weaker leading to fading spectral peaks.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A device for the detection of kinase and/or phosphatase activity said device comprising:
a Raman active surface comprising features that enhance Raman scattering;
said surface having attached thereto at lease one kinase and/or phosphatase substrate molecule.

2. The device of claim 1, where said surface has attached thereto a plurality of kinase and/or phosphatase substrate molecules.

3. The device of claim 2, wherein said kinase substrate molecules are selected from the group consisting of a small molecule, a lipid, a peptide, a phosphorylated small molecule, a phosphorylated lipid, a phosphorylated peptide, a nucleotide, a sugar, a polysaccharide, a polymer, a lipids, a phosphorylated nucleotide, a phosphorylated sugar, a phosphorylated polysaccharide, a phosphorylated polymer, and a phosphorylated lipid.

4-7. (canceled)

8. The device of claim 3, wherein said substrate is a peptide substrate for a kinase selected from the group consisting of a serine kinase, threonine kinase, histidine kinase, and a tyrosine kinase.

9. (canceled)

10. The device of claim 2, wherein said plurality of peptides comprises at least 5 different peptides.

11-12. (canceled)

13. The device of claim 2, wherein the length of said peptides ranges from about 5 to about 50 amino acids.

14. The device of claim 10, wherein said peptides are localized such that signals from each species of peptide are distinguishable from signals from the other species of peptide.

15. The device of claim 2, wherein the features that enhance Raman scattering comprise a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers.

16. The device of claim 2, wherein the features that enhance Raman scattering comprise a material selected from the group consisting of a metal, a carbon-based material, a polymer, a quartz material, a liquid crystal material, a metal oxide material, a salt crystal, a semiconductor material, a noble metal, a noble metal alloy, and a noble metal composite.

17. (canceled)

18. The device of claim 2, wherein the features that enhance Raman scattering comprise a material selected from the group consisting of gold, gold alloy, silver, silver alloy, copper, copper alloy, platinum, platinum alloy, CdSe semiconductor, CdS semiconductor, CdSe coated with ZnS, magnetic colloidal materials, ZnS, ZnO, TiO₂, AgI, AgBr, HgI₂, PbS, PbSe, ZnTe, CdTe, In₂S3, In₂Se₃, Cd₃P₂, Cd₃As₂, InAs, and GaAs.

19. (canceled)

20. The device of claim 2, wherein the center to center distance of said features ranges from about 25 nm to about 0.5 μm.

21-23. (canceled)

24. The device of claim 1, wherein said Raman active surface comprises or is disposed within a microfluidic chamber.

25. The device of claim 1, wherein said surface has attached thereto a plurality of kinase and/or phosphatase substrate molecules.

26. The device of claim 25, wherein said plurality of kinase and/or phosphatase substrate molecules comprises at least 5 species.

27. (canceled)

28. The device of claim 24, wherein the volume of said chamber is less than about 1 μL.

29. The device of claim 1, wherein:
the Raman active surface comprises gold nanopyramids;
the kinase substrate molecule comprises a plurality of protein kinase and/or phosphatase substrates; and
the Raman active surface comprises or is disposed within a microfluidic chamber.

30. A method of detecting and/or quantifying kinase and/or phosphatase activity in a sample, said method comprising:
contacting said sample with a molecule comprising a kinase and/or phosphatase substrate sequence; and
detecting phosphorylation or dephosphorylation of said molecule by detecting a change in the Raman scattering spectrum of said peptide.

31. The method of claim 30, wherein said kinase substrate molecules are selected from the group consisting of a small molecule, a lipid, a peptide, a phosphorylated small molecule, a phosphorylated lipid, a phosphorylated peptide, a nucleotide, a sugars, a polysaccharide, a polymer, a lipid, phosphorylated nucleotides, a phosphorylated sugar, a phosphorylated polysaccharide, a phosphorylated polymer, and a phosphorylated lipid.

32-35. (canceled)

36. The method of claim 30, wherein said substrate molecules are substrates for a kinase selected from the group consisting of a serine kinase, a threonine kinase, a histidine kinase, and a tyrosine kinase.

37. (canceled)

38. The method of claim 30, wherein said kinase or phosphatase substrate molecule is attached to a Raman active surface comprising features that enhance Raman scattering.

39. The method of claim 38, where said surface has attached thereto a plurality of kinase and/or phosphatase substrate molecules.

40. The method of claim 39, wherein said plurality of kinase and/or phosphatase substrate molecules comprises at least 5 species.

41-42. (canceled)

43. The method of claim 39, wherein said kinase and/or phosphatase substrate molecules are localized such that signals from each species of kinase and/or phosphatase substrate are distinguishable from signals from the other species of kinase substrate.

44. The method of claim 2, wherein the features that enhance Raman scattering comprises a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers.

45. The method of claim 38, wherein the features that enhance Raman scattering comprise a metal or semiconductor material.

46-48. (canceled)

49. The method of claim 38, wherein the center to center distance of said features ranges from about 25 to about 500 nm.

50. (canceled)

51. The method of claim 38, wherein the features that enhance Raman scattering have a size that ranges from about 20 nm to about 200 nm.

52. (canceled)

53. The method of claim 38, wherein said Raman active surface comprises or is disposed within a microfluidic chamber.

54. The method of claim 38, wherein the volume of said chamber is less than about 1 μL.

55. The method of claim 38, wherein:
the Raman active surface comprises gold nanopyramids;
the kinase substrate molecule comprises a plurality of tyrosine kinase substrates; and
the Raman active surface comprises or is disposed within a microfluidic chamber.

56. A system for the detection of kinase and/or phosphatase activity in one or more samples, said system comprising:
a device according to claim 1; and
a Raman detection probe disposed to measure surface enhanced Raman spectra from one or more regions of said device.

57-58. (canceled)

59. The system of claim 56, wherein said Raman detection probe comprises a laser light delivery fiber, an objective lens, a long-pass optical filter, and a Raman scattering light collection fiber.

60. (canceled)

61. A method of screening a sample for a modulator of kinase and/or phosphatase activity, said method comprising:
contacting a device according to claim 1 with a test sample containing one or more test agents;
performing a SERS measurement to detect a change in the Raman scattering spectrum when the kinase and/or phosphatase substrates are phosphorylated or dephosphorylated, where an inhibition in change of the Raman spectrum indicates that a test agent is an inhibitor of kinase or phosphatase activity.

62-63. (canceled)

64. A method of making a surface for detection of kinase and/or phosphatase activity, said method comprising
depositing an array of kinase and/or phosphatase substrate molecules on a first surface;
contacting said array of kinase and/or phosphatase substrate molecules with a SERS surface comprising a plurality of features that enhance Raman scattering, wherein said contacting is under conditions that transfer the kinase and/or phosphatase substrate molecules from said first surface to said SERS surface to form a surface for the detection of kinase and/or phosphatase activity.

65. The method of claim 64, wherein said kinase and/or phosphatase substrate molecules bear a functional group or a linker having a functional group that reacts to form a covalent linkage with the SERS surface.

66. The method of claim 64, wherein said SERS surface is formed on a soft-lithographic substrate.

67. (canceled)

68. The method of claim 64, further comprising disposing said SERs surface in or attaching said SERs surface to a microfluidic structure to form a well adjacent to the SERS surface.

69. (canceled)

70. The method of claim 64, wherein the array of kinase and/or phosphatase substrate molecules comprises a spacing between dots that ranges from about 20 to about 500 nm.

71-80. (canceled)

81. The method of claim 64, wherein said kinase and/or phosphatase substrate molecules are localized such that signals from each species of peptide are distinguishable from signals from the other species of peptide.

82. The method of claim 64, wherein the features that enhance Raman scattering comprises a multiplicity of nanoscale features selected from the group consisting of nanoscale pyramids, nanoscale dots, nanoscale fibers, nanotubes, nanohorns, nanoholes, nano bowties, nanobowls, nanocrescents, and nanoburgers.

83-90. (canceled)

91. A method of fabricating a nanopyramid surface, said method comprising:
providing a photolithographable surface;
contacting the surface with a first plasma to produce a nanoscale oxide island array;
etching the surface to form a nanopillar array;
removing the oxide layer on the nanopillars comprising the nanopillar array;
etching the nanopillar array to form a nanopyramid array.

92. The method of claim 91, wherein said method further comprises metalizing said nanopyramid array.

93. The method of claim 91, wherein said photolithographable surface comprises a silicon or germanium surface.

94-104. (canceled)
